(19) United States
(12) Patent Application Publication
(10) Pub. No.: US 2023/0256196 A1
(43) Pub. Date: Aug. 17, 2023

(54) A Hybrid Bioscaffold-Intravascular Catheter for Cellular Therapies

(71) Applicant: The Board of Trustees of the Leland Stanford Junior University, Stanford, CA (US)

(72) Inventors: Avnesh S. Thakor, Palo Alto, CA (US); Sophia Hu, Redwood City, CA (US); Rosita Primavera, Redwood City, CA (US); Mehdi Razavi, Palo Alto, CA (US)

(21) Appl. No.: 18/014,616

(22) PCT Filed: Jul. 21, 2021
(86) PCT No.: PCT/US2021/042539

§ 371 Date: Jan. 5, 2023

Related U.S. Application Data
(60) Provisional application No. 63/055,136, filed on Jul. 22, 2020.

(51) Int. Cl. A61M 25/00; C08J 9/26

(52) U.S. Cl. CPC A61M 25/0045; A61M 25/007; A61M 25/0009; C08J 9/26; A61M 2025/006; A61M 2210/12; A61L 29/005

(57) ABSTRACT

An intravascular multi-side hole catheter containing a bioscaffold capable of housing therapeutic cells is provided. The catheter comprises a plurality of side holes distributed along the length of the catheter in a spiraling corkscrew pattern. The bioscaffold inside the catheter is designed with a plurality of macropores capable of encapsulating therapeutic cells for cellular therapy. Upon placement of the catheter in a vein, the side holes allow blood to flow though the catheter thereby supplying oxygen and nutrients to any loaded cellular cargo and also providing for the removal of waste products. Methods of producing the intravascular catheter and methods of using the intravascular catheter in cellular therapy, including for delivery of insulin-secreting cells such as beta cells or stem cell-derived islets into blood vessels for treating type 1 diabetes are also disclosed.

### BACKGROUND OF THE INVENTION

Type-1 diabetes (T1D) is caused by immune mediated destruction of insulin-producing beta-cells resulting in insulin deficiency and hyperglycemia (WHO| Diabetes. WHO. 2017). In the United States, T1D is among the top 10 causes of death and is estimated to cost the US healthcare system over 245 billion dollars each year. The incidence of T1D has dramatically increased over the past 50 years, with approximately 30.3 million Americans estimated to have this disease (Center for Disease Control. National Diabetes Statistic Report, 2017. CDC.gov.cdc.gov/features/diabetes-statistic-report/index.html. Published 2017. Accessed Mar. 1, 2018). The treatment for T1 D can be broadly classified into 3 groups: 1) pharmacological therapies (i.e. daily injections of insulin); 2) artificial pancreas-like systems (i.e. closed-loop feedback of insulin); and 3) cellular therapies (i.e. islet transplantation) (Primavera et al. (2020) Nanomaterials 10(4):789). Although insulin injections are the most straightforward treatment, they require a regimented schedule and cannot respond dynamically to changes in blood glucose concentrations. To account for this, various artificial pancreas-like systems have been developed to mimic the physiological response to blood glucose changes. These systems rely on a closed-loop feedback of insulin to maintain the concentration of glucose within a narrow range (Veiseh et al. (2015) Nat. Rev. Drug Discov. 14(1):45-57, Chu (2005) Expert Opin. Ther. Pat. 15(9):1147-1155). However, this has not been widely adopted as hoped as many of these artificial pancreas-like systems require a consistent source of exogenous insulin in addition to periodic replacement of the enzymatic glucose sensor (Chu, supra; Di et al. (2014) Adv. Healthc. Mater. 3(6):811-816).

An alternative treatment for T1 D is islet transplantation, which offers a biological cellular therapy whereby donor islets are given to a recipient patient with T1D (Fiorina et al. (2008) Am. J. Transplant. 8(10):1990-1997, Liew et al. (2014) Curr. Diab. Rep. 14(3):469). The main advantage of islet transplantation relies on the fact that patients can now endogenously produce insulin in real time, with islets responding dynamically to changes in blood glucose levels to maintain euglycemia. Currently, allogenic islet transplantation for the treatment of T1D is still undergoing clinical trials in the US despite being approved in other countries (Foster et al. (2018) Diabetes Care 41(5):1001-1008). However, the combination of limited pancreatic islet donors, the autoimmune reaction to donor islets, the liver being a relatively hypoxic site for transplantation, and the instant blood-mediated inflammatory reaction results in approximately 60% of transplanted islets being immediately lost following transplantation (Muthyala et al. (2017) Xenotransplantation 24(1):e12275, Geleto et al. (2016) Ethiop. J. Health Sci. 26(3):237-242, Goldstick et al. (1976) Adv. Exp. Med. Biol. 75:183-190).

Therefore, it would be highly beneficial to develop devices and methods for transplanting islets that would improve their survival and function.

### SUMMARY OF THE INVENTION

An intravascular multi-side hole catheter containing a bioscaffold capable of housing therapeutic cells is provided. The catheter comprises a plurality of side holes distributed along the length of the catheter in a spiraling corkscrew pattern. The bioscaffold inside the catheter is designed with a plurality of macropores capable of encapsulating therapeutic cells for cellular therapy. Upon placement of the catheter in a vein, the side holes allow blood to flow though the catheter thereby supplying oxygen and nutrients to any loaded cellular cargo and also providing for the removal of waste products. Methods of producing the intravascular catheter and methods of using the intravascular catheter in cellular therapy, including for delivery of insulin-secreting cells such as beta cells or stem cell-derived islets into blood vessels for treating type 1 diabetes are also disclosed.

In one aspect, an intravascular catheter is provided, the catheter comprising: a) a catheter tube comprising a lumen and a plurality of side holes, wherein the side holes are distributed along the length of the catheter tube in a spiraling corkscrew pattern; and b) a biocompatible bioscaffold comprising a plurality of macropores and micropores, wherein the biocompatible bioscaffold is contained within the catheter lumen.

In certain embodiments, the catheter tube comprises polyurethane or silicone.

In certain embodiments, the bioscaffold comprises a polymer. Exemplary polymers include, without limitation, polydimethylsiloxane (PDMS), collagen, albumin, fibrin, alginate, graphene, nylon (polyamides), dacron (polyesters), polystyrene, polypropylene, poly(acrylic acids), poly(methacrylic acids), polyvinyl compounds (e.g., polyvinyl chloride, polyvinyl acetate), polycarbonate (PC), poly(alkylene oxides), polyvinylpyrrolidone (PVP), polytetrafluorethylene (PTFE, teflon), thermanox (TPX), polymers of hydroxy acids, such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof.

In certain embodiments, the bioscaffold further comprises a coating comprising an anticoagulant (e.g., heparin, warfarin or other coumarins), for example, to reduce the formation of blood clots/thrombus within the catheter.

In certain embodiments, the catheter tube has a French size ranging from 3 french to 34 french, including any French size in this range such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In some embodiments, the catheter has a French size ranging from 3 french to 20 french.

In certain embodiments, the side holes are about 0.2 to about 5.0 mm wide, about 3 mm to about 10 mm apart, and rotated about 30° to about 60° between each side hole. In some embodiments, the side holes are about 5.0 mm wide, about 10 mm apart, and rotated about 60° between each side hole. In other embodiments, the side holes are about 0.2 mm wide, about 3.0 mm apart and rotated about 30° between each hole.

In certain embodiments, the bioscaffold has a porosity ranging from about 30% to about 95%, including any porosity within this range such as 30%, 35%, 40%, 45%, 50%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. In some embodiments, the porosity is about 80 percent.

In certain embodiments, the macropores have an average diameter ranging from about 150 to 800 micrometers, including any average diameter within this range, such as 150, 155, 160, 165, 170, 175, 180, 185, 190, 200, 225, 250, 255, 260, 265, 270, 275, 280, 285, 290, 295, 300, 305, 310, 315, 320, 325, 330, 335, 340, 345, 350, 355, 360, 365, 370, 375, 380, 400, 420, 440, 460, 480, 500, 520, 540, 560, 580, 600, 620, 640, 660, 680, 700, 720, 740, 760, 780, or 800 μm. In some embodiments, the macropores have an average diameter ranging from about 150 to about 250 μm

In certain embodiments, the micropores have an average diameter of 30 μm or less.

In certain embodiments, the bioscaffold further comprises therapeutic cells, wherein the therapeutic cells are contained in the macropores. In some embodiments, the therapeutic cells are stem cells (e.g., induced-pluripotent stem cells, embryonic stem cells, or adult stem cells), progenitor cells, or mature cells. In some embodiments, the therapeutic cells secrete a cytokine, a chemokine, a growth factor, or a hormone. In some embodiments, the therapeutic cells are endocrine cells. In some embodiments, the therapeutic cells are genetically modified.

In certain embodiments, the therapeutic cells are insulin-secreting cells. For example, the insulin-secreting cells may be pancreatic beta cells or islets obtained from a donor. Alternatively, the insulin-secreting cells may be derived from stem cells or pancreatic progenitor cells.

In certain embodiments, the bioscaffold further comprises one or more agents, including without limitation, drugs, growth factors, angiogenic agents, cytokines, or extracellular matrix components, or a combination thereof.

In another aspect, a method of implanting therapeutic cells in a subject is provided, the method comprising placing an intravascular catheter, described herein, within a major vein of the subject. Exemplary major veins include without limitation, an internal jugular vein, a subclavian vein, and a femoral vein.

In certain embodiments, the therapeutic cells are autologous, allogeneic, or xenogeneic.

In certain embodiments, the method further comprises retrieving the intravascular catheter from the subject. In some embodiments, the method further comprises exchanging the intravascular catheter for another intravascular catheter comprising therapeutic cells.

In another aspect, a method of treating a subject for type 1 diabetes is provided, the method comprising placing an intravascular catheter comprising a bioscaffold comprising insulin-secreting cells within a major vein of the subject.

In certain embodiments, the insulin-secreting cells are autologous, allogeneic, or xenogeneic pancreatic beta cells or islets.

In certain embodiments, the insulin-secreting cells are derived from stem cells or pancreatic progenitor cells.

In certain embodiments, the method further comprises retrieving the intravascular catheter from the subject and exchanging the intravascular catheter for another intravascular catheter comprising insulin-secreting cells.

In another aspect, a kit comprising an intravascular catheter described herein and packaging comprising a compartment for holding the intravascular catheter is provided.

In certain embodiments, the kit further comprises therapeutic cells.

In certain embodiments, the therapeutic cells are contained in the macropores of the biocompatible bioscaffold or in a container separate from the intravascular catheter.

In some embodiments, the therapeutic cells are stem cells (e.g., induced-pluripotent stem cells, embryonic stem cells, or adult stem cells), progenitor cells, or mature cells. In some embodiments, the therapeutic cells secrete a cytokine, a chemokine, a growth factor, or a hormone. In some embodiments, the therapeutic cells are endocrine cells. In some embodiments, the therapeutic cells are genetically modified.

In certain embodiments, the therapeutic cells are insulin-secreting cells. For example, the insulin-secreting cells may be pancreatic beta cells or islets obtained from a donor. Alternatively, the insulin-secreting cells may be derived from stem cells or pancreatic progenitor cells.

In certain embodiments, the kit further comprises instructions for using the intravascular catheter.

In another aspect, a method of producing an intravascular catheter described herein is provided, the method comprising: a) combining polydimethylsiloxane (PDMS) with a porogen to produce a PDMS-porogen mixture; b) placing the PDMS-porogen mixture inside the catheter lumen; c) curing the PDMS-porogen mixture inside the catheter lumen; d) adding a plurality of side holes to the catheter tube, wherein the side holes are distributed along the length of the catheter tube in a spiraling corkscrew pattern; and e) removing the porogen from the PDMS-porogen mixture, wherein a PDMS bioscaffold comprising a plurality of macropores and micropores is produced inside the catheter lumen.

In certain embodiments, the porogen is water soluble. In some embodiments, the porogen is an alkali metal salt, for example, including without limitation, sodium chloride or potassium chloride.

In certain embodiments, removing the porogen comprises submerging the intravascular catheter in water.

In certain embodiments, the method further comprises adding therapeutic cells to the bioscaffold, wherein the therapeutic cells are contained in the macropores.

In certain embodiments, the catheter tube has a French size ranging from 3 french to 34 french, including any French size in this range such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In some embodiments, the catheter tube has a French size ranging from 3 french to 20 french.

In certain embodiments, the side holes are about 0.2 to about 5.0 mm wide, about 3 mm to about 10 mm apart, and rotated about 30° to about 60° between each side hole. In some embodiments, the side holes are about 5.0 mm wide, about 10 mm apart, and rotated about 60° between each side hole. In other embodiments, the side holes are about 0.2 mm wide, about 3.0 mm apart and rotated about 30° between each hole.

In certain embodiments, the bioscaffold has a porosity ranging from about 30% to about 95%, including any porosity within this range such as 30%, 35%, 40%, 45%, 50%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. In some embodiments, the porosity is about 80 percent.

In certain embodiments, the macropores have an average diameter ranging from about 150 to 800 micrometers, including any average diameter within this range, such as 150, 155, 160, 165, 170, 175, 180, 185, 190, 200, 225, 250, 255, 260, 265, 270, 275, 280, 285, 290, 295, 300, 305, 310, 315, 320, 325, 330, 335, 340, 345, 350, 355, 360, 365, 370, 375, 380, 400, 420, 440, 460, 480, 500, 520, 540, 560, 580, 600, 620, 640, 660, 680, 700, 720, 740, 760, 780, or 800 μm. In some embodiments, the macropores have an average diameter ranging from about 150 to about 250 μm

In certain embodiments, the catheter comprises polyurethane or silicone.

In another aspect, a method of producing an intravascular catheter is provided, the method comprising: forming a catheter tube comprising a plurality of side holes using three-dimensional (3D) printing, wherein the side holes are distributed along the length of the catheter tube in a spiraling corkscrew pattern; and forming a biocompatible bioscaffold using the 3D printing, wherein the biocompatible bioscaffold is contained inside the catheter lumen.

In certain embodiments, 3D-printing is used to generate the catheter tube and the biocompatible scaffold together or separately.

In certain embodiments, the bioscaffold comprises a geometric lattice inside the catheter tube.

In certain embodiments, the bioscaffold comprises a plurality of macropores and micropores. In certain embodiments, the macropores have an average diameter ranging from about 150 to 800 micrometers, including any average diameter within this range, such as 150, 155, 160, 165, 170, 175, 180, 185, 190, 200, 225, 250, 255, 260, 265, 270, 275, 280, 285, 290, 295, 300, 305, 310, 315, 320, 325, 330, 335, 340, 345, 350, 355, 360, 365, 370, 375, 380, 400, 420, 440, 460, 480, 500, 520, 540, 560, 580, 600, 620, 640, 660, 680, 700, 720, 740, 760, 780, or 800 μm. In some embodiments, the macropores have an average diameter ranging from about 150 to about 250 μm. In certain embodiments, the micropores have an average diameter of 30 μm or less.

In certain embodiments, the bioscaffold has a porosity ranging from about 30% to about 95%, including any porosity within this range such as 30%, 35%, 40%, 45%, 50%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. In some embodiments, the porosity is about 80 percent.

In certain embodiments, the catheter tube has a French size ranging from 3 french to 34 french, including any French size in this range such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34. In some embodiments, the catheter tube has a French size ranging from 3 french to 20 french.

In certain embodiments, the side holes are about 0.2 to about 5.0 mm wide, about 3 mm to about 10 mm apart, and rotated about 30° to about 60° between each side hole. In some embodiments, the side holes are about 5.0 mm wide, about 10 mm apart, and rotated about 60° between each side hole. In other embodiments, the side holes are about 0.2 mm wide, about 3.0 mm apart and rotated about 30° between each hole.

In certain embodiments, the method further comprising adding therapeutic cells to the bioscaffold.

In certain embodiments, the method further comprises designing a model of the intravascular catheter using computer aided designing (CAD) software to control production of the intravascular catheter by 3D printing.

### BRIEF DESCRIPTION OF THE DRAWINGS

FIG. 1. Schematic illustration of the hybrid PDMS bioscaffold-intravascular catheter in the bloodstream. In this schematic, the catheter is placed in the internal jugular vein (IJV). Catheter side holes allow blood entry into the catheter, where islets (or other cellular therapy cargo) are housed in a PDMS bioscaffold. Insulin (as well as waste products) can then be secreted by islets back into the systemic circulation.

FIGS. 2A-2J. (left) CFD analysis of the catheters to determine hole orientation. FIGS. 2A-2B) CFD simulation images of the 14F and 4F catheter. Arrows indicate velocity vectors and color indicates the speed of the vector, ranging from dark blue (slowest velocity) to bright red (fastest velocity). Catheters were modeled using Solidworks using an extruded hollow cylinder (the catheter) within a larger extruded cylinder (the blood vessel) and a bioscaffold matrix was approximated as a resistance within the inner catheter. Side hole orientation were simulated for the 14F and 4F catheter: a linear. Holes (linear), two linear lines of holes placed opposite each other (parallel-linear), and holes that twist around the catheter in a corkscrew pattern (corkscrew). Linear, parallel-linear, and corkscrew hole designs were assessed for both the 14F and 4F catheter to determine hole orientation. FIGS. 2C-2D) Water age and FIGS. 2E-2F) oxygen diffusion across the length of the catheters (14F and 4F, respectively). The corkscrew hole orientation was determined to best minimize water age and maximize oxygen diffusion in both catheters. (right) Side hole optimization of 14F and 4F catheters to determine the optimal side hole diameter, distance, and angle of rotation using the corkscrew orientation. FIGS. 2G-2H) Water age and FIG. 2I-2J) oxygen diffusion were assessed across the length of the catheters (14F and 4F catheter, respectively). Data labels are expressed as (hole diameter)-(angle of rotation)-(distance between holes).

FIGS. 3A-3B. SEM images of PDMS bioscaffolds at different porosities within the catheter. PDMS bioscaffolds were developed in silicon catheters via solvent casting and particulate leaching technique at 30%, 50%, 60%, 70%, and 80% porosities (n=3). FIG. 3A) SEM images at two different two different magnifications, 75×(top row) and 150×(bottom row), reveal that 80% was the optimal porosity for the bioscaffold. There was minimal separation between the catheter walls and the interconnected pores. 80% porosity was used for all in vitro experiments. FIG. 3B) Plot of pore size versus porosity.

FIGS. 4A-4E. In vitro assessment of the catheter containing the PDMS bioscaffold. FIGS. 4A-4B) Fluorescent microscopy images of the islets only and islets within the catheter containing PDMS bioscaffold. White arrows indicate islets loaded in the bioscaffold within the catheter. Green (FDA staining) and red (PI staining) fluorescence indicate live and dead cells, respectively. FIG. 4C) Islet viability was evaluated after 24 h of incubation. There was no significant difference in viability between any of the groups. FIG. 4D) Functionality of islets was assessed with a GSIS assay after 24 h of incubation time at different glucose conditions. Functionality is reported as the amount of insulin secretion (ng/mL) in low (2.3 mM of glucose) and high glucose (20.0 mM of glucose) conditions. FIG. 4E) Stimulation indices calculated by dividing the insulin concentration in islets exposed at high glucose levels (20.0 mM glucose, HG) to those produced in a low glucose environment (2.3 mM glucose, LG-2). Results are expressed as the average ±SEM, and the statistical analysis was conducted using Student's t-test (d) or one-way ANOVA with post-hoc Tukey test (FIG. 4E). Significant results are expressed as * and a, considering any differences statistically significant when p<0.05. *p<0.05 low glucose (LG-1 or LG-2) vs. High glucose (HG) for each group; ^{a}p<0.05 indicates results significant when comparing the control vs. PDMS bioscaffold only and catheter+PDMS bioscaffold groups.

FIG. 5. Meshed model (14F).

FIG. 6. Meshed model (4F).

### DETAILED DESCRIPTION

An intravascular multi-side hole catheter containing a bioscaffold capable of housing therapeutic cells is provided. The catheter comprises a plurality of side holes distributed along the length of the catheter in a spiraling corkscrew pattern. The interior bioscaffold of the catheter is designed with a plurality of macropores capable of encapsulating therapeutic cells for cellular therapy. Upon placement of the catheter in a vein, the side holes allow blood to flow though the catheter thereby supplying oxygen and nutrients to any loaded cellular cargo and also providing for the removal of waste products. Methods of producing the intravascular catheter and methods of using the intravascular catheter in cellular therapy, including for delivery of insulin-secreting cells such as beta cells or stem cell-derived islets into blood vessels for treating type 1 diabetes are also disclosed.

Before the intravascular multi-side hole catheter and methods of producing it and using it in cellular therapy are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “a cell” includes a plurality of such cells and reference to “the polymer” includes reference to one or more polymers and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

“Biocompatible,” as used herein, refers to a property of a material that allows for prolonged contact with a tissue in a subject without causing toxicity or significant damage.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

“Active agent” and “drug” are used interchangeably to refer to any chemical compound that can have a therapeutic and/or preventive effect for a disease when suitably administered to a subject.

“Therapeutically effective amount” refers to an amount effective, at dosages and for periods of time necessary, to achieve the desired therapeutic result.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as Induced pluripotent stem cells can be derived, for example, from adult somatic cells such as peripheral blood mononuclear cells, fibroblasts, keratinocytes, epithelial cells, endothelial progenitor cells, mesenchymal stem cells, adipose cells, leukocytes, hematopoietic stem cells, bone marrow cells, or hepatocytes.

“Substantially” as used herein, may be applied to modify any quantitative representation that could permissibly vary without resulting in a change in the basic function to which it is related. For example, a scaffold may have dimensions that deviate somewhat from being flat, if the cell encapsulation and/or tissue graft properties of the scaffold is not materially altered.

“Diameter” as used in reference to a shaped structure (e.g., macropore, micropore, cell aggregate, etc.) refers to a length that is representative of the overall size of the structure. The length may in general be approximated by the diameter of a circle of sphere that circumscribes the structure.

As used herein, the term “cell viability” refers to a measure of the number of cells that are living or dead, based on a total cell sample. High cell viability, as defined herein, refers to a cell population in which greater than 80% of all cells are viable, preferably greater than 90-95%, and more preferably a population characterized by high cell viability containing more than 97-99% viable cells.

“Substantially purified” generally refers to isolation of a substance (e.g., compound, polynucleotide, protein, polypeptide, antibody, aptamer) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with hyperglycemia or pre-diabetic) as well as those in which prevention is desired (e.g., those with increased susceptibility to diabetes, those having a genetic predisposition to developing diabetes, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

As used herein, the term “cell viability” refers to a measure of the number of cells that are living or dead, based on a total cell sample. High cell viability, as defined herein, refers to a cell population in which greater than 80% of all cells are viable, preferably greater than 90-95%, and more preferably a population characterized by high cell viability containing more than 97-99% viable cells.

### An Intravascular Catheter Containing a Bioscaffold for Implantation of Therapeutic Cells

The present disclosure pertains to an intravascular catheter containing a bioscaffold for implantation of therapeutic cells. The catheter tube comprises a plurality of side holes distributed along the length of the catheter in a spiraling corkscrew pattern. The bioscaffold inside the catheter lumen is designed with a plurality of macropores capable of encapsulating therapeutic cells for cellular therapy. The intravascular catheter is designed to be placed directly into the bloodstream. Upon placement of the catheter in a vein, the side holes allow blood to flow though the catheter thereby supplying oxygen and nutrients to any loaded cellular cargo and also providing for the removal of waste products.

The catheter may be composed of various polymers including, without limitation, silicone, polyurethane, polyethylene terephthalate, latex, nylon, polyimides, and thermoplastic elastomers. A single lumen or a multi-lumen (e.g., double, or triple lumen) catheter may be used. The use of a multi-lumen catheter allows different intravenous infusions to be connected to each lumen using one catheter access site. In some embodiments, it may be desirable to include an additional lumen, for example, to allow administration of drugs through the catheter into the vein in addition to providing the therapeutic cells.

In certain embodiments, the catheter tube has a French size ranging from 3 french to 34 french, from 10 french to 16 french, or from 3 french to 8 french, or any size within these ranges such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 french. In some embodiments, the catheter tube has a French size ranging from 3 french to 20 french.

In certain embodiments, the side holes are about 0.2 mm to about 5.0 mm wide, about 3 mm to about 10 mm apart, and rotated about 30° to about 60° between each side hole. These parameters can be optimized for catheters with lumens of different sizes to provide the greatest flow into and out of the catheter, minimize water age, and maximize oxygen diffusion in the catheter (see Examples). For example, a 14F catheter can be used with the side holes about 5.0 mm wide, about 10 mm apart, and rotated about 60° between each side hole. In another example, a 4F catheter can be used with the side holes about 0.2 mm wide, about 3.0 mm apart and rotated about 30° between each hole.

In some embodiments, the side holes have a width ranging from 0.2 mm to about 5.0 mm, including any width within this range, such as 0.2 mm, 0.3 mm, 0.4 mm, 0.5 mm, 0.6 mm, 0.7 mm, 0.8 mm, 0.9 mm, 1.0 mm, 1.2 mm, 1.4 mm, 1.6 mm, 1.8 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5.0 mm.

In some embodiments, the side holes are spaced apart by a distance ranging from about 3 mm to about 10 mm, including any distance within this range, such as 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

In some embodiments, the side holes are distributed along the catheter tube with a rotation of about 30° to about 60° between each side hole, including any degree of rotation within this range, such as 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, or 60°.

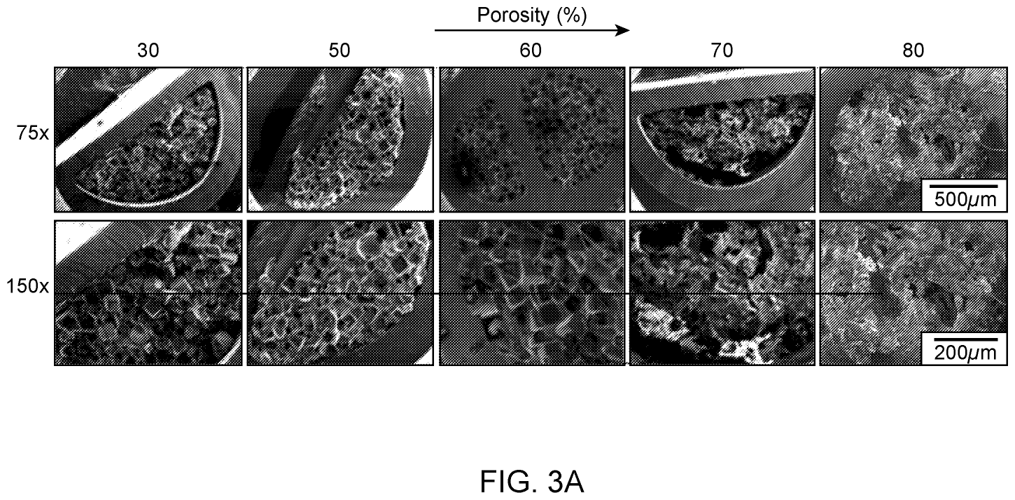

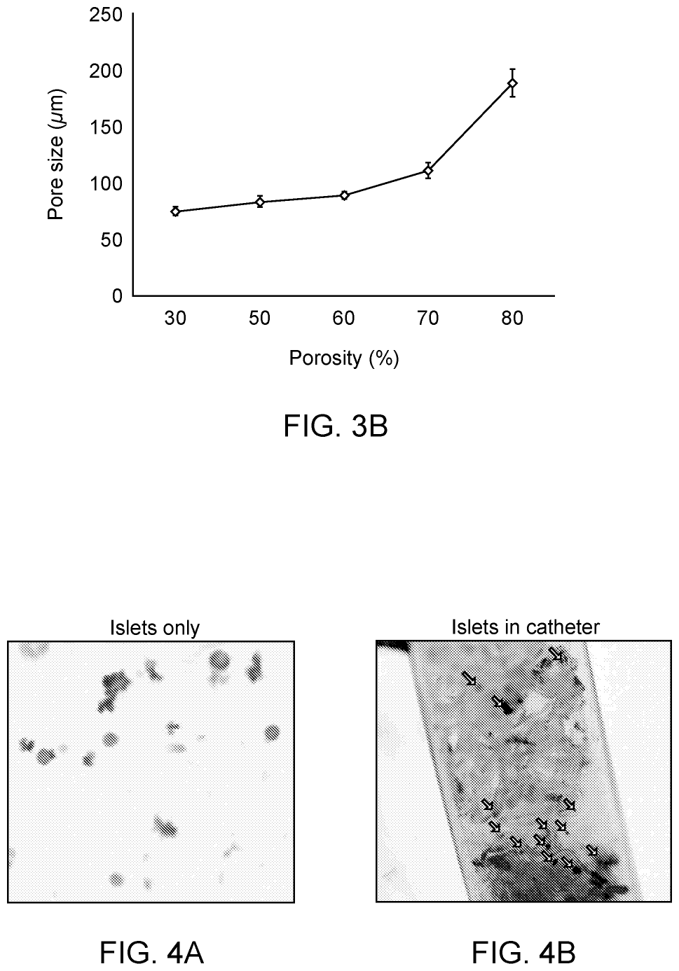

The bioscaffold provides an environment, e.g., microenvironment, for various types of cells, such as therapeutic cells for use in cellular therapy, to attach and grow therein. The bioscaffold is synthesized within the catheter lumen using a solvent casting and particulate leaching technique (see Example 2). Accordingly, the bioscaffold adopts the shape of the catheter lumen and helps to distribute the therapeutic cells along the length of the catheter. Preferably, the bioscaffold provides a structure that allows therapeutic cells to be evenly distributed throughout the catheter. The bioscaffold structure contains pores, including macropores and micropores (see FIG. 3A). The macropores generally have an average diameter large enough to accommodate therapeutic cells or cell aggregates.

In some embodiments, the macropores have an average diameter ranging from about 150 to 800 micrometers, including any average diameter within this range, such as 150, 155, 160, 165, 170, 175, 180, 185, 190, 200, 225, 250, 255, 260, 265, 270, 275, 280, 285, 290, 295, 300, 305, 310, 315, 320, 325, 330, 335, 340, 345, 350, 355, 360, 365, 370, 375, 380, 400, 420, 440, 460, 480, 500, 520, 540, 560, 580, 600, 620, 640, 660, 680, 700, 720, 740, 760, 780, or 800 mm. In some embodiments, the macropores have a diameter of about 50 μm or more, e.g., about 100 μm or more, about 150 μm or more, about 200 μm or more, about 250 μm or more, about 300 μm or more, including about 350 μm or more, and may have a diameter of about 800 μm or less, e.g., about 500 μm or less, about 400 μm or less, about 300 μm or less, about 250 μm or less, including about 200 μm or less. In some embodiments, the macropores have an average diameter ranging from about 150 to about 250 μm

In some embodiments, the micropores have an average diameter of about 40 μm or less, e.g., about 38 μm or less, about 36 μm or less, about 34 μm or less, about 32 μm or less, about 30 μm or less, about 28 μm or less, about 26 μm or less, about 24 μm or less, about 22 μm or less, about 20 μm or less, about 18 μm or less, about 16 μm or less, about 14 μm or less, about 12 μm or less, including about 10 μm or less. In some embodiments, the micropores have an average diameter in the range of about 1 μm to about 50 μm, e.g., about 10 μm to about 40 μm, including about 20.0 μm to about 30 μm. In certain embodiments, the micropores have an average diameter of 30 μm or less.

The bioscaffold has a suitable porosity for supporting growth and/or maintenance of cells encapsulated therein. In some embodiments, the bioscaffold has a porosity of about 50% or more, e.g., about 55% or more, about 60% or more, about 65% or more, about 70% or more, including about 75% or more, and in some cases, has a bulk porosity of about 95% or less, e.g., about 90% or less, about 85% or less, about 80% or less, including about 75% or less. In some embodiments, the scaffold has a porosity in the range of about 50% to about 95%, e.g., about 55% to about 90%, about 60% to about 85%, e.g., about 65% to about 80%, including any porosity within these ranges, such as 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95%. In some cases, the bioscaffold has a porosity of about 75% to about 85%. In some embodiments, the bioscaffold has a porosity of about 80%. The porosity may be measured, for example, using scanning electron microscopy (SEM) or computed tomography (CT) scanning.

In certain embodiments, the bioscaffold comprises a polymer. Exemplary polymers include, without limitation, polydimethylsiloxane (PDMS), collagen, albumin, fibrin, alginate, graphene, nylon (polyamides), dacron (polyesters), polystyrene, polypropylene, poly(acrylic acids), poly(methacrylic acids), polyvinyl compounds (e.g., polyvinyl chloride, polyvinyl acetate), polycarbonate (PC), poly(alkylene oxides), polyvinylpyrrolidone (PVP), polytetrafluorethylene (PTFE, teflon), thermanox (TPX), polymers of hydroxy acids, such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof.

In certain embodiments, the bioscaffold further comprises a coating comprising an anticoagulant (e.g., heparin, warfarin or other coumarins), for example, to reduce the formation of blood clots/thrombus within the catheter.

A bioscaffold having therapeutic cells encapsulated therein may provide a carrier for transplanting cells by implanting the intravascular catheter containing the bioscaffold in a blood vessel. The therapeutic cells may be encapsulated within the macropores of the bioscaffold. In some cases, where the cells grow as aggregates (where two or more cells are attached to one another) when grown in a conventional culture environment (e.g., grown on a two-dimensional culture dish or flask surface), the macropores have an average diameter that approximates the average size of the cell aggregates and the cell aggregates are encapsulated within the macropores. In other cases, individual cells are distributed in the macropores throughout the bioscaffold.

In some cases, the bioscaffold may provide for the maintenance or growth as well as oxygenation of the cells cultured therein for a time period (e.g., one day or more, two days or more, 3 days or more, 4 days or more, 5 days or more, 6 days or more, 8 days or more, 10 days or more, 1 week or more, 2 weeks or more, 3 weeks or more) in in vitro culture. Thus, cells encapsulated in the bioscaffold may at least maintain the same number of cells, or may expand by two times or more, e.g., 3 times or more, 4 times or more, 5 times or more, 10 times or more, 20 times or more 50 times or more, including 100 times or more in number after a time period in culture as compared to the number of cells initially seeded on the scaffold. In some cases, cells encapsulated in the bioscaffold expands by a range of 2 to 1000-fold, e.g., 2 to 100-fold, 2 to 50-fold, including 3 to 20-fold in number after the time period in culture.

The intravascular catheter of the present disclosure containing therapeutic cells encapsulated in a bioscaffold may maintain the cells in a functional state suitable for providing a therapeutic effect (e.g., insulin secretion by beta-cells) when implanted into a blood vessel. The therapeutic cells may maintain responsiveness to physiological cues (e.g., blood glucose level). Without wishing to be bound by theory, it is thought that the porosity (i.e., the macro- and micro-porosity) of the bioscaffold inside the lumen of the intravascular catheter presents to the encapsulated cells a microenvironment that provides desirable nutrient transport and vascular integration to grow and maintain the cells in a functional state. Implantation of the catheter containing the bioscaffold in a blood vessel provides therapeutic cells with access to the bloodstream for the supply of oxygen and nutrients and the removal of waste products.

The therapeutic cells are, in some cases, stably encapsulated within the bioscaffold such that the cells remain in the bioscaffold when the catheter is implanted in a blood vessel of an individual. In some cases, about 20% or less, e.g., about 15% or less, about 10% or less, about 5% or less, about 2% or less, about 1% or less, about 0.1% or less, about 0.01% or less, including about 0.001% or less of the total number of therapeutic cells encapsulated in the bioscaffold may exit the bioscaffold when implanted in a blood vessel of an individual.

In some cases, the therapeutic cells can migrate out of the bioscaffold when the intravascular catheter is implanted in a blood vessel of an individual. The extent to and/or rate at which therapeutic cells exit the bioscaffold when the intravascular catheter is implanted in a blood vessel of an individual may vary, and may depend on a variety of controllable factors, such as the size of the pores (macropores and/or micropores), the size of the pores (macropores and/or micropores) relative to the size of the cells or cell aggregates encapsulated in the bioscaffold, the degradation rate of the bioscaffold matrix, the density of the bioscaffold matrix, the porosity of the bioscaffold matrix, surface modification of the bioscaffold, etc.

The bioscaffold within the catheter may have encapsulated therein any suitable amount of the therapeutic cells. The amount of cells may depend on a variety of factors, such as the function provided by the therapeutic cells, the size of the catheter lumen, the length of time the catheter is to be implanted, the condition to be treated by the therapeutic cells and/or the desired therapeutic outcome. In some cases, the bioscaffold comprises at least 10⁵ cells, e.g., at least 10⁶ cells, at least 10⁷ cells, at least 10⁸ cells, at least 10⁹ cells, at least 10¹⁰ cells, or more cells encapsulated therein.

In some embodiments, individual cells are distributed in the macropores throughout the bioscaffold matrix, which may improve cell survival and function by preventing clumping of cells. In other embodiments, the therapeutic cells are aggregating cells. Aggregating cells may be cells that, when grown on the surface of a culture dish or in suspension, attach to one another to form clumps (i.e., aggregates) of two or more cells, e.g., 10 or more cells, 100 or more cells, 1,000 or more cells, including 10,000 or more cells. The aggregate of cells may also be attached to a solid support (e.g., the culture dish surface) or may be free-floating in the medium. The aggregate of cells may be any suitable shape, and in some cases, may be spherical or oval. The size of the aggregate may be any suitable size. In some cases, the cell aggregate that forms in a conventional culture condition (e.g., in suspension, or on a two-dimensional surface) has an average diameter that approximates the average diameter of the macropores of the present bioscaffold. Thus, in some cases, the therapeutic cells may form, in a conventional culture condition, cell aggregates having an average diameter that is within about 50%, e.g., within about 40%, within about 30%, within about 20%, within about 10%, including within about 5% of the average diameter of the macropores of the bioscaffold inside the lumen of the intravascular catheter. In some embodiments, the therapeutic cells may form, in a conventional culture condition, cell aggregates having an average diameter of about 50 μm or more, e.g., about 75 μm or more, about 100 μm or more, including about 125 μm or more, and in some cases, an average diameter of about 300 μm or less, e.g., about 275 μm or less, about 250 μm or less, about 225 μm or less, including about 200 μm or less. In some cases, the therapeutic cells may form, in a conventional culture condition, cell aggregates having an average diameter in the range of about 50 μm to about 300 μm, e.g., about 75 μm to about 275 μm, about 75 μm to about 250 μm, about 100 μm to about 225 μm, including about 100 μm to about 200 μm.

The aggregate of cells may be a collection of a substantially pure population of cells or may be a collection of a plurality of cell-types, e.g., two more types, three or more types, four or more types, including 5 or more types of cells. In some cases, the aggregate of cells is stem cell-derived. In some cases, the aggregate of cells is an embryoid body that includes pluripotent stem cells and/or cells differentiated therefrom.

In some embodiments, the therapeutic cells include cells that secrete a biological agent, e.g., a signaling molecule, a hormone, a growth factor, a cytokine, a chemokine, an enzyme, an antibody, etc. Such cells may include, without limitation, pancreatic islets, pancreatic beta cells, hepatocytes, thyroid follicular and parafollicular cells, adrenal cortical cells, parathyroid chief cells, or immune cells. In some cases, the therapeutic cells include cells whose activity is conditional, e.g., cells that modulate their function based on the physiological state of the host, such as glucose level in the blood and/or the environment of the host tissue. The therapeutic cell may be a type of cell that specifically possesses the functional activity by virtue of its cell type (e.g., by differentiating or having differentiated into a cell type that exhibits the functional activity), or may be genetically modified to exhibit the functional activity that was not exhibited by the cell before being genetically modified.

Exemplary therapeutic molecules that can be secreted by a therapeutic cell include, without limitation, insulin, human growth hormone, thyroxine, glucagon-like peptide-1 (GLP-1), GLP-1 (7-37), GLP-1 (7-36), and like GLP-1 receptor agonist polypeptides, GLP-2, interleukins 1 to 33 (e.g., IL-1, IL-2, IL-3, IL-4, IL-5, IL-6, IL-7, IL-8, IL-9, IL-10, IL-11, IL-12, IL-13, IL-17, IL-18, IL-21, IL-22, IL-27, IL-33), interferon (α, β, γ), GM-CSF, G-CSF, M-CSF, SCF, FAS ligands, TRAIL, leptin, adiponectin, blood coagulation factor VIII/blood coagulation factor IX, von Willebrand factor, glucocerebrosidase, lipoprotein lipase (LPL), lecithin-cholesterol acyltransferase (LCAT), erythropoietin, apoA-I, albumin, atrial natriuretic peptide (ANP), luteinizing hormone releasing hormone (LHRH), angiostatin/endostatin, endogenous opioid peptides (enkephalins, endorphins, etc.), calcitonin/bone morphogenetic protein (BMP), pancreatic secretory trypsin inhibitors, catalase, superoxide dismutase, anti-TNF-α antibody, soluble IL-6 receptor, IL-1 receptor antagonist, α2 antitrypsin, etc.

The therapeutic cells may be any suitable type of cell for transplanting to an individual in need. The therapeutic cells may be stem cells, progenitor cells, or mature cells. The cells may be autologous, allogeneic, xenogeneic or genetically-modified.

In some cases, the therapeutic cells are stem cell-derived cells. Stem cells of interest include, without limitation, hematopoietic stem cells, embryonic stem cells, adult stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, induced pluripotent stem cells; and the like; as well as differentiated cells that can be cultured in vitro and used in a therapeutic regimen, where such cells include, but are not limited to, keratinocytes, adipocytes, cardiomyocytes, neurons, osteoblasts, pancreatic islet cells, retinal cells, and the like. The cell that is used will depend in part on the nature of the disorder or condition to be treated.

Suitable human embryonic stem (ES) cells include, but are not limited to, any of a variety of available human ES lines, e.g., BG01 (hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA09/Oct4D10 (H9-hOct4-pGZ), WA13 (H13), WA14 (H14) (Wisconsin Alumni Research Foundation; WARF; Madison, Wis.). Cell line designations are given as the National Institutes of Health (NIII) code, followed in parentheses by the provider code.

Hematopoietic stem cells (HSCs) are mesoderm-derived cells that can be isolated from bone marrow, blood, cord blood, fetal liver and yolk sac. HSCs are characterized as CD34⁺ and CD3⁻. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

Mesenchymal stem cells (MSCs) can be obtained from connective tissue including, without limitation, bone marrow, placenta, umbilical cord blood, adipose tissue, muscle, corneal stroma, and dental pulp of deciduous baby teeth. MSCs can differentiate to form muscle, bone, cartilage, fat, marrow stroma, and tendon. Methods of isolating MSCs are known in the art; and any known method can be used to obtain MSCs.

An induced pluripotent stem (iPS) cell is a pluripotent stem cell induced from a somatic cell, e.g., a differentiated somatic cell. iPS cells are capable of self-renewal and differentiation into cell fate-committed stem cells, including hormone-secreting, growth factor-secreting, or cytokine-secreting cells, as well as various types of mature cells. iPS cells can be generated from somatic cells, including, without limitation, peripheral blood mononuclear cells, fibroblasts, keratinocytes, epithelial cells, endothelial progenitor cells, mesenchymal stem cells, adipose cells, leukocytes, hematopoietic stem cells, bone marrow cells, and hepatocytes, using, e.g., known methods. iPS cells can be generated from somatic cells by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and K1f4. In some embodiments, somatic cells are genetically modified with one or more expression constructs comprising nucleotide sequences encoding Oct-4, Sox2, Nanog, and LIN28. Methods of generating iPS are known in the art, and any such method can be used to generate iPS.

In some cases, the therapeutic cells are lymphocytes, such as CD4+ and/or CD8+ T lymphocytes, or B lymphocytes. In some embodiments, the therapeutic cells are plasma B cells. In some embodiments, the therapeutic cells are cytotoxic T lymphocytes. In some embodiments, the lymphocytes are genetically modified lymphocytes, e.g., chimeric antigen receptor (CAR) T lymphocytes.

In some embodiments, the therapeutic cells include insulin-secreting cells. The insulin-secreting cells may be any suitable type of insulin-secreting cell. In some cases, the insulin-secreting cells are a type of cell that secretes insulin (e.g., pancreatic β islet cells, or β-like cells). In some cases, the insulin-secreting cells are primary β islet cells (e.g., mature β islet cells isolated from a pancreas). In some cases, the insulin-secreting cells are β cells, or β-like cells that are derived in vitro from immature cells, precursor cells, progenitor cells, or stem cells. The insulin-secreting cells may be derived from (i.e., obtained by differentiating) stem and/or progenitor cells such as hepatocytes (e.g., transdifferentiated hepatocytes), acinar cells, pancreatic duct cells, stem cells, embryonic stem cells (ES), partially differentiated stem cells, non-pluripotent stem cells, pluripotent stem cells, induced pluripotent stem cells (iPS cells), etc. Suitable insulin-secreting cells and methods of generating the same are described in, e.g., US20030082810; US20120141436; and Raikwar et al. (PLoS One. 2015 Jan. 28; 10(1):e0116582), each of which are incorporated herein by reference.

The insulin-secreting cells may produce (e.g., secrete) insulin at a rate independent of the ambient/extracellular glucose concentration (e.g., the concentration of glucose in the host tissue in which the tissue graft is implanted), or may produce (e.g., secrete) insulin at a rate that depends on the ambient/extracellular glucose concentration. In some cases, the insulin-secreting cells constitutively secrete insulin. In some embodiments, the insulin-secreting cells increase insulin secretion when the ambient/extracellular glucose concentration increases, and decrease insulin secretion when the ambient/extracellular glucose concentration decreases.

A suitable coating may be included on the bioscaffold (e.g., on the surface of the macropores and/or micropores of the scaffold) to promote encapsulation of the therapeutic cells. The coating may include a biological coating (e.g., extracellular matrix proteins) and/or may include a synthetic coating (such as described in US20070032882, which is incorporated herein by reference). A suitable biological coating includes extracellular matrix proteins, such as, without limitation, collagen, fibronectin, vitronectin, laminin, heparan sulfate, proteoglycan, glycosaminoglycan, chondroitin sulfate, hyaluronan, dermatan sulfate, keratin sulfate, elastin, and combinations thereof.

In some embodiments, one or more active agents are adsorbed or absorbed within the bioscaffold. In some embodiments, the active agent is an immunosuppressant, such as, but not limited to cyclosporine and tacrolimus. In some cases, the active agent is an inhibitor of the mammalian target of rapamycin (mTOR), such as, without limitation, rapamycin and analogs thereof (e.g., sirolimus, temsirolimus, everolimus, deforolimus, etc.). The mTOR inhibitor may be used as an immunosuppressant, or may be an anticancer agent. In some cases, the active agent is a binding agent, such as an antibody, or an antigen binding fragment thereof. The antibody may be any suitable antibody that specifically binds to an antigen expressed by a therapeutic cell of interest for encapsulating in the present scaffolds. Suitable antigens include, without limitation, CD3, CD28, CD137, CTLA-4, TNF, IL-6, IL-12, PD-1, PD-L1, TIM3, LAGS, IL-2Ralpha, IL-23, IL-6R, CD25, IL-17, IL-1, CD4, CD8, LFA-1, IL-22, and IL-20.

Other suitable active agents according to embodiments of the present disclosure may include but are not limited to interferon, interleukin, erythropoietin, granulocyte-colony stimulating factor (GCSF), stem cell factor (SCI:), leptin (OB protein), interferon (alpha, beta, gamma), antibiotics such as vancomycin, gentamicin ciprofloxacin, amoxycillin, lactobacillus, cefotaxime, levofloxacin, cefipime, mebendazole, ampicillin, lactobacillus, cloxacillin, norfloxacin, tinidazole, cefpodoxime, proxctil, azithromycin, gatifloxacin, roxithromycin, cephalosporin, anti-thrombogenics, aspirin, ticlopidine, sulfinpyrazone, heparin, warfarin, growth factors, differentiation factors, hepatocyte stimulating factor, plasmacytoma growth factor, glial derived neurotrophic factor (GDNF), neurotrophic factor 3 (NT3), fibroblast growth factor (FGF), transforming growth factor (TGF), platelet transforming growth factor, milk growth factor, endothelial growth factors, endothelial cell-derived growth factors (ECDGF), alpha-endothelial growth factors, beta-endothelial growth factor, neurotrophic growth factor, nerve growth factor (NGF), vascular endothelial growth factor (VEGF), 4-1 BB receptor (4-IBBR), TRAIL (TNF-related apoptosis inducing ligand), artemin (GFRalpha3-RET ligand), BCA-I (B cell-attracting chemokinel), B lymphocyte chemoattractant (BLC), B cell maturation protein (BCMA), brain-derived neurotrophic factor (BDNF), bone growth factor such as osteoprotegerin (OPG), bone-derived growth factor, thrombopoietin, megakaryocyte derived growth factor (MDGF), keratinocyte growth factor (KGF), platelet-derived growth factor (PDGF), ciliary neurotrophic factor (CNTF), neurotrophin 4 (NT4), granulocyte colony-stimulating factor (GCSF), macrophage colony-stimulating factor (mCSF), bone morphogenetic protein 2 (BMP2), BRAK, C-IO, Cardiotrophin 1 (CTI), CCR8, anti-inflammatory: paracetamol, salsalate, diflunisal, mefenamic acid, diclofenac, piroxicam, ketoprofen, dipyrone, acetylsalicylic acid, anti-cancer drugs such as alitretinoin, altretamine, anastrozole, azathioprine, bicalutarnide, busulfan, capecitabine, carboplatin, cisplatin, cyclophosphamide, cytarabine, doxorubicin, epirubicin, etoposide, exemestane, vincristine, vinorelbine, hormones, thyroid stimulating hormone (TSH), sex hormone binding globulin (SHBG), prolactin, luteotropic hormone (LTH), lactogenic hormone, parathyroid hormone (PTH), melanin concentrating hormone (MCH), luteinizing hormone (LHb), growth hormone (HGH), follicle stimulating hormone (FSHb), haloperidol, indomethacin, doxorubicin, epirubicin, amphotericin B, Taxol, cyclophosphamide, cisplatin, methotrexate, pyrene, amphotericin B, anti-dyskinesia agents, Alzheimer vaccine, antiparkinson agents, ions, edetic acid, nutrients, glucocorticoids, heparin, anticoagulation agents, antivirus agents, anti-HIV agents, polyamine, histamine and derivatives thereof, cystineamine and derivatives thereof, diphenhydramine and derivatives, orphenadrine and derivatives, muscarinic antagonist, phenoxybenzamine and derivatives thereof, protein A, streptavidin, amino acid, beta-galactosidase, methylene blue, protein kinases, beta-amyloid, lipopolysaccharides, eukaryotic initiation factor-4G, tumor necrosis factor (TNF), tumor necrosis factor-binding protein (TNF-bp), interleukin-1 (to 18) receptor antagonist (IL-Ira), granulocyte macrophage colony stimulating factor (GM-CSF), novel erythropoiesis stimulating protein (NESP), thrombopoietin, tissue plasminogen activator (TPA), urokinase, streptokinase, kallikrein, insulin, steroid, acetaminophen, analgesics, antitumor preparations, anti-cancer preparations, anti-proliferative preparations or pro-apoptotic preparations, among other types of active agents.

In some embodiments, the one or more absorbed active agents is a compound selected from the group consisting of chemotactic agents, cell attachment mediators, integrin binding sequences, epidermal growth factor (EGF), hepatocyte growth factor (HGF), vascular endothelial growth factors (VEGF), fibroblast growth factors, platelet derived growth factors (PDGF), insulin-like growth factor, transforming growth factors (TGF), parathyroid hormone, parathyroid hormone related peptide, bone morphogenetic proteins (BMP), BMP-2, BMP-4, BMP-6, BMP-7, BMP-12, BMP-13, BMP-14, transcription factors, growth differentiation factor (GDF), GDF5, GDF6, GDF8, recombinant human growth factors, cartilage-derived morphogenetic proteins (CDMP), CDMP-1, CDMP-2 and CDMP-3.

Methods of Transplanting Cells into an Individual

Also provided herein is a method of transplanting cells into an individual, using an intravascular catheter comprising a bioscaffold containing therapeutic cells, as described herein, e.g., to treat a disease. The method may include implanting (e.g., surgically implanting) the intravascular catheter comprising the bioscaffold containing the therapeutic cells into a major blood vessel of an individual. In some embodiments, the intravascular catheter is placed in a major vein of the patient, such as an internal jugular vein, a subclavian vein, or a femoral vein. Intravascular catheters can be implanted into patients by using surgical techniques well-known in the art, including surgical laparoscopy or laparotomy. Exemplary methods include without limitation the Seldinger technique, the modified Seldinger technique, the Trocar technique, and the Moncrief-Popovich technique. Additionally, intravascular catheters may be implanted using ultrasound guidance to identify the target site (e.g., major vein) and guide puncture and catheter placement. In some cases, implanting the intravascular catheter may be performed in conjunction with another therapy, such as another surgical operation and/or administration of a drug.

The host individual may be suffering from a condition, e.g., a disease, that may be treated by providing the therapeutic cells to the individual. In some cases, the disease is diabetes (type 1 or type 2). In some cases, the individual has pre-diabetes, or hyperglycemia. In some cases, the disease is cancer (e.g., breast cancer, prostate cancer, brain cancer, skin cancer, lung cancer, liver cancer, colorectal cancer, etc.). The therapeutic cells may be any suitable therapeutic cells, as described above, and the type of therapeutic cells may depend on the disease to be treated.

Also provided herein is a method of regulating blood glucose levels in an individual using an intravascular catheter containing a bioscaffold comprising insulin-secreting cells, as described herein. The present method may include implanting the intravascular catheter comprising a bioscaffold containing insulin-secreting cells encapsulated in its pores (e.g., macropores), as described herein, into a major blood vessel (e.g., an internal jugular vein, a subclavian vein, or a femoral vein) of a host individual, to maintain normoglycemia in the individual. The individual may be suffering from dysregulation of blood glucose, and may have, e.g., type 1 or type 2 diabetes, pre-diabetes, or hyperglycemia. The insulin-secreting cells may be any suitable insulin-secreting cells, as described above. The bioscaffold within the intravascular catheter may include any suitable number of cells, as described above, and in some cases includes 10⁵ to 10⁹ cells, e.g., 10⁶ to 10⁸ cells. The present method may further comprise culturing the insulin-secreting cells on the bioscaffold, as described herein, to encapsulate the insulin-secreting cells in the macropores of the bioscaffold.

A medical practitioner may locate the blood vessel for implantation of the intravascular catheter comprising a bioscaffold containing therapeutic cells, for example, by medical imaging (e.g. ultrasound, radiography, or MRI). In some embodiments, a contrast agent is included in the composition comprising the therapeutic cells to allow confirmation of the location of the cells by medical imaging after implantation. In some embodiments, the contrast agent is a microbubble (e.g., for use in ultrasound) or a radiopaque contrast agent (e.g., for use in radiography). The contrast agent may be contained in the same composition as the therapeutic cells or in a different composition and used prior to or after implantation of the intravascular catheter comprising a bioscaffold containing therapeutic cells.

### Methods of Making an Intravascular Catheter Comprising a Bioscaffold

Also provided herein is a method of making an intravascular catheter comprising a bioscaffold contained inside the catheter lumen, as described herein. In general terms, the present method may include combining a solution of a polymer in a solvent with a porogen. A “porogen” as used herein may refer to any material that can be incorporated into a matrix to reserve a space in the matrix, which can be removed from the matrix to generate a pore. The amount of the porogen used in the mixture depends on the desired porosity of the bioscaffold.

The bioscaffold can be fabricated from polymers including, without limitation, polydimethylsiloxane (PDMS), collagen, albumin, fibrin, alginate, graphene, nylon (polyamides), dacron (polyesters), polystyrene, polypropylene, poly(acrylic acids), poly(methacrylic acids), polyvinyl compounds (e.g., polyvinyl chloride, polyvinyl acetate), polycarbonate (PC), poly(alkylene oxides), polyvinylpyrrolidone (PVP), polytetrafluorethylene (PTFE, teflon), thermanox (TPX), polymers of hydroxy acids, such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof.

The mixture containing the polymer and the porogen is placed inside the catheter lumen. The solvent may be removed (e.g., by evaporation) from the deposited mixture, to form the bioscaffold matrix inside the catheter lumen. The porogen then may be removed from the bioscaffold matrix using any suitable method, depending on the type of porogen used to produce the porous (e.g., microporous and macroporous) bioscaffold. In some cases, the mixture of the starting polymer material and solvent is incubated at an elevated temperature (e.g., at a range of 50° C. to 90° C., e.g., 60° C. to 80° C., including 65° C. to 75° C.) for a sufficient period of time (e.g., 1 hour or longer, 2 hours or longer, 3 hours or longer, 4 hours or longer, 6 hours or longer, 8 hours or longer, 10 hours or longer, including 12 hours or longer, 1 day or longer, 2 days or longer, 3 days or longer, 4 days or longer, 5 days or longer, 6 days or longer, 1 week or longer, or 2 weeks or longer) for curing.

After curing, side holes are added to the catheter tube using any suitable method. For example, the side holes can be created by puncturing the catheter with a soldering tip or other implement suitable for creating holes of the desired width. The side holes should be distributed along the length of the catheter in a spiraling corkscrew pattern. In certain embodiments, the side holes are about 0.2 mm to about 5.0 mm wide, about 3 mm to about 10 mm apart, and rotated about 30° to about 60° between each side hole.

In some embodiments, side holes are added to the catheter having a width ranging from 0.2 mm to about 5.0 mm, including any width within this range, such as 0.2 mm, 0.3 mm, 0.4 mm, 0.5 mm, 0.6 mm, 0.7 mm, 0.8 mm, 0.9 mm, 1.0 mm, 1.2 mm, 1.4 mm, 1.6 mm, 1.8 mm, 2.0 mm, 2.2 mm, 2.4 mm, 2.6 mm, 2.8 mm, 3.0 mm, 3.2 mm, 3.4 mm, 3.6 mm, 3.8 mm, 4.0 mm, 4.2 mm, 4.4 mm, 4.6 mm, 4.8 mm, or 5.0 mm.

In some embodiments, the side holes are spaced apart by a distance ranging from about 3 mm to about 10 mm, including any distance within this range, such as 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, or 10 mm.

In some embodiments, the side holes are distributed along the catheter tube with a rotation of about 30° to about 60° between each side hole, including any degree of rotation within this range, such as 30°, 31°, 32°, 33°, 34°, 35°, 38°, 37°, 38°, 39°, 40°, 41°, 42°, 43°, 44°, 45°, 48°, 47°, 48°, 49°, 50°, 51°, 52°, 53°, 54°, 55°, 56°, 57°, 58°, 590, or 60°.

These parameters can be optimized for catheters with lumens of different sizes to provide the greatest flow into and out of the catheter, minimize water age, and maximize oxygen diffusion in the catheter (see Examples). For example, a 14F catheter can be used with the side holes about 5.0 mm wide, about 10 mm apart, and rotated about 60° between each side hole. In another example, a 4F catheter can be used with the side holes about 0.2 mm wide, about 3.0 mm apart and rotated about 30° between each hole.

The porogen may be any suitable material (e.g., chemical substance) that can be incorporated into the matrix to reserve a space and be removed from the scaffold to form pores in the matrix. In some embodiments, the porogen is water soluble. Water soluble porogens of interest include water soluble salts, such as an alkali metal salt, an alkali earth metal salt, ammonium salt; sugars, polysaccharides; and water soluble synthetic or natural polymers. In some cases, the water-soluble salt is a chloride salt. In some cases, the alkali metal salt is sodium chloride or potassium chloride. In some embodiments, the porogen is a heat-labile compound, such as sodium bicarbonate or hydrogen peroxide.

The porogen may be a particulate material, where the particles of the porogen remain suspended in the mixture of the polymer and porogen. The particulate porogen may have any suitable particle size, where the particle size may depend on the desired size of the macropores in the bioscaffold. The porogen particle size may be similar to the size of the macropores. In some embodiments, the porogen particles have an average diameter of about 50 μm or more, e.g., about 60 μm or more, about 70 μm or more, about 80 μm or more, about 90 μm or more, including about 100 μm or more, and may have an average diameter of about 500 μm or less, e.g., about 400 μm or less, about 300 μm or less, about 250 μm or less, including about 200 μm or less. In some embodiments, the porogen particles have an average diameter in the range of about 50 μm to about 500 μm, e.g., about 60 μm to about 400 μm, about 70 μm to about 300 μm, about 80 μm to about 250 μm, about 90 μm to about 200, including about 150 μm to about 250 μm.

The amount of porogen added to form the mixture of the polymer and porogen may be any suitable amount, which may depend on the desired porosity of macropores in the bioscaffold. In some embodiments, the amount of particulate porogen (e.g., NaCl) added is about 10⁴ particles/g polymer in solution or more, e.g., about 3×10⁴ particles/g polymer in solution or more, about 6×10⁴ particles/g polymer in solution or more, about 10⁵ particles/g polymer in solution or more, about 3×10⁵ particles/g polymer in solution or more, including about 6×10⁵ particles/g polymer in solution or more, and in some embodiments, is about 10⁹ particles/g polymer in solution or less, e.g., about 6×10⁸ particles/g polymer in solution or les, 3×10⁸ particles/g polymer in solution or less, about 10⁸ particles/g polymer in solution or less, about 6×10⁷ particles/g polymer in solution or less, about 3×10⁷ particles/g polymer in solution or less, about 10⁷ particles/g polymer in solution or less, about 6×10⁶ particles/g polymer in solution or less, including about 3×10⁶ particles/g polymer in solution or less. In some embodiments, the amount of particulate porogen (e.g., NaCl) added is from about 10⁴ particles/g polymer in solution to about 10⁹ particles/g polymer in solution, e.g., from about 3×10⁴ particles/g polymer in solution to about 6×10⁸ particles/g polymer in solution, from about 6×10⁴ particles/g polymer in solution to about 3×10⁸ particles/g polymer in solution, from about 10⁵ particles/g polymer in solution to about 10⁸ particles/g polymer in solution, from about 10⁵ particles/g polymer in solution to about 6×10⁷ particles/g polymer in solution, from about 10⁵ particles/g polymer in solution to about 3×10⁷ particles/g polymer in solution, including from about 10⁵ particles/g polymer in solution to about 10⁷ particles/g polymer in solution.

In some embodiments, the amount of NaCl added as porogen to the polymer solution is about 0.1 g/g polymer in solution or more, e.g., 0.5 g/g polymer in solution or more, 1.0 g/g polymer in solution or more, 2.0 g/g polymer in solution or more, 3.0 g/g polymer in solution or more, including 4.0 g/g polymer in solution or more, and in some cases, is about 50 g/g polymer in solution or less, e.g., 40 g/g polymer in solution or less, 30 g/g polymer in solution or less, 20 g/g polymer in solution or less, including 10 g/g polymer in solution or less. In some embodiments, the amount of NaCl added as porogen to the polymer solution is from about 0.1 g/g polymer in solution to about 50 g/g polymer in solution, e.g., from about 0.5 g/g polymer in solution to about 40 g/g polymer in solution, from about 1.0 g/g polymer in solution to about 30 g/g polymer in solution, from about 2.0 g/g polymer in solution to about 20 g/g polymer in solution, from about 3.0 g/g polymer in solution to about 20 g/g polymer in solution, including from about 4.0 g/g polymer in solution to about 10 g/g polymer in solution.

The mixture of the polymer and porogen may be inserted into the catheter lumen using any suitable method. In some embodiments, the catheter is a commercially available catheter having a French size ranging from 3 french to 20 french. In other embodiments, the catheter is produced using a mold or cast.

Removing the solvent may be done using any suitable method. In some cases, removing the solvent includes evaporating the solvent. In some cases, evaporating the solvent includes exposing the polymer-porogen mixture to a vacuum, heat, convective flow of a gas (e.g., air, nitrogen, argon, etc.) or a combination thereof. In some cases, the solvent is evaporated by lyophilizing the deposited mixture. Removing the solvent may be done for a sufficient period of time (e.g., 1 hour or longer, 2 hours or longer, 3 hours or longer, 4 hours or longer, 6 hours or longer, 8 hours or longer, 10 hours or longer, including 12 hours or longer) to remove 90% or more, e.g., 95% or more, 97% or more, 99% or more, or substantially all the solvent from the mixture.

The porogen may be removed from the matrix using any suitable method, depending on the porogen used. In some cases, removing the porogen includes submerging the polymer matrix in an aqueous solution (e.g., distilled water) to leach out the porogen, e.g., if the porogen is a water-soluble salt. In some cases, removing the porogen includes exposing the polymer matrix to heat, if the porogen is heat labile. Removing the porogen may be done for a sufficient period of time (e.g., 1 hour or longer, 3 hours or longer, 6 hours or longer, 12 hours or longer, 1 day or longer, 3 days or longer, 5 days or longer, including 7 days or longer). The length of time may depend on the type of porogen used and the method of removal. After the removal step, 80% or more, e.g., 85% or more, 90% or more, 95% or more, 97% or more, or substantially all the porogen may be removed from the polymer matrix.

Alternatively, three-dimensional (3D) printing can be used to generate the intravascular catheter. 3D printing generally involves the fabrication of structures through successive layer deposition controlled by a computerized process. Computer aided designing (CAD) software can be used to design a model of the bioscaffold or a model of a catheter tube containing a bioscaffold for fabrication. The model of the bioscaffold may be designed to include macropores and micropores of a specified size, porosity, and interconnectivity. In some embodiments, 3D-printing is used to generate a geometric lattice within a tube, which can function as an intravascular catheter that can be used in cell therapy, as described herein. Any suitable 3D printing technique may be used, including, without limitation, fused deposition modeling (FDM), selective laser sintering (SLS), stereolithography (SLA), electrospinning, and bio-electrospraying. The bioscaffold can be produced by 3D-printing with various polymers, including, without limitation, poly(ethylene glycol) diacrylate (PEGDA) and natural gelatin methacrylate (GeIMA), poly(ε-caprolactone) (PCL), poly(D, L-lactic-co-glycolic acid) (PLGA) scaffolds, polyglycolic acid (PGA), poly(propylene fumarate) (PPF) and poly(hydroxy butyrate)(PHB), collagen, corn starch, dextran, alginate, and chitosan, and others discussed below. The choice of polymer may depend on the particular 3D printing method used. For a review of 3D printing techniques, see, e.g., Zhu et al. (2016) Curr. Opin. Biotechnol. 40:103-112, Jammalamadaka et al. (2018) J. Funct. Biomater. 9(1):22, and Do et al. (2015) Adv. Healthc. Mater. 4(12):1742-62.

In some embodiments, fused deposition modeling (FDM) is used. FDM is a 3D printing technique that utilizes thermoresponsive polymers such as, but not limited to, acrylonitrile butadiene styrene (ABS), polycarbonate (PC), polyetherimide (PEI), polyphenylsulfone (PPSF), polylactic acid (PLA), polyethylene terephthalate glycol (PETG), polyethylene terephthalate (PET), high-impact polystyrene (HIPS), thermoplastic polyurethane (TPU), and aliphatic polyamides (nylon). See, e.g., Hamzah et al. (2018) Electrochemistry Communications 96: 27-371; U.S. Pat. No. 5,121,329; Bardot et al. (2020) Nanomaterials 10(12):2567; Wasti et al. (2020) Front Chem. 8:315; Chua et al. (2003) Rapid Prototyping: Principles and Applications. Singapore: World Scientific. p. 124.

In some embodiments, the selective laser sintering (SLS) method is used. SLS is a 3D printing technique that involves sintering powdered particles together in a specified pattern using a laser beam (e.g., a CO₂ laser). SLS can be performed with polymers such as, but not limited to, polycaprolactone, polyvinyl alcohol, poly (lactic acid), nylon, polyamides (PA), polystyrenes (PS), thermoplastic elastomers (TPE), and polyaryletherketones (PAEK). See, e.g., U.S. Pat. No. 4,863,538; Mazzoli (2013) Med. Biol. Eng Comput. 51(3):245-256; Shirazi et al. (2015) Sci Technol Adv Mater. 16(3):033502.

In some embodiments, stereolithography (SLA) is used. SLA is a 3D printing technique that can be used to form a scaffold from a photosensitive polymer. A photosensitive polymer is deposited as a liquid onto a surface and exposed to light (e.g., UV range of 300-400 nm). This process is repeated until the scaffold is completed. SLA can be performed with polymers such as, but not limited to, polypropylene fumarate (PPF) with photocrosslinkable bonds and polyethylene glycol acrylate. See, e.g., Crivello et al. (2014) “Photopolymer Materials and Processes for Advanced Technologies.” Chemistry of Materials Chem. Mater. 26.1: 533; Melchels et al. (2010) Biomaterials 31(24):6121-6130; Skoog et al. (2014) J. Mater. Sci. Mater. Med. 25(3):845-856; Chartrain et al. (2018) Acta Biomater. 74:90-111.

In some embodiments, electrospinning is used. Electrospinning produces fibers from polymer solutions using electrostatic forces. Electrospinning can be performed using synthetic or natural polymers such as, but not limited to, poly(ε-caprolactone), collagen, chitosan, and gelatin. See, e.g., Merritt et al. (2012) Advanced Engineering Materials. 14 (5): B266-B278; Xue et al. (2019) Chem Rev. 119(8):5298-5415; Hong et al. (2019) Int. J. Mol. Sci. 20(24):6208; Muerza-Cascante et al. (2015) Tissue Eng Part B Rev. 21(2):187-202.

In some embodiments, bio-electrospraying is used. Bio-electrospraying is similar to electrospinning, but deposits small droplets instead of fine fibers. See, e.g., Suwan et al. (2007) Materials Today. 10 (6): 60; Maurmann et al. (2018) Adv. Exp. Med. Biol. 1078:79-100.

As discussed herein, therapeutic cells may be encapsulated in the macropores of the bioscaffold (e.g., produced with a porogen or 3D-printing). Thus, provided herein is a method of making a porous (e.g., microporous and macroporous) bioscaffold having therapeutic cells encapsulated in the macropores of the bioscaffold. In general terms, the method may include depositing cells of interest on the scaffold, and culturing in vitro the deposited cells for a sufficient time under suitable conditions to maintain, expand and/or differentiate the cells, thereby providing an effective amount of therapeutic cells encapsulated in the macropores of the bioscaffold.

In some embodiments, where the cells are aggregating cells, as described above, the method may include pre-culturing a first population of the cells in conventional culture conditions (e.g., on a two dimensional culture dish surface, or in suspension), to produce a cell aggregate, loosening the cell aggregate, and depositing the loosened cell aggregate onto the polymeric scaffold.

The number of cells that are encapsulated in the bioscaffold after the culturing in the present method may be at least as much as the number of cells that are initially deposited on the scaffold. In some embodiments, the present method expands the number of cells initially deposited on the scaffold by about 1.2 fold or more, e.g., about 1.5 fold or more, about 2.0 fold or more, about 3.0 fold or more, about 5.0 fold or more, about 10 fold or more, about 20 fold or more, about 30 fold or more, about 50 fold or more, about 100 fold or more, about 200 fold or more, about 500 fold or more, about 1,000 fold or more, including about 10,000 fold or more to produce the encapsulated cells. In some embodiments, the present method expands the number of cells initially deposited on the scaffold by a range of about 1.2 fold to about 1.5 fold, about 1.5 fold to about 2.0 fold, about 2.0 fold to about 3.0 fold, about 3.0 fold to about 5.0 fold, about 5.0 fold to about 10 fold, about 10 fold to about 20 fold, about 20 fold to about 30 fold, about 30 fold to about 50 fold, about 50 fold to about 100 fold, about 100 fold to about 200 fold, about 200 fold to about 500 fold, about 500 fold to about 1,000 fold, or about 1,000 fold to about 10,000 fold.

The number of cells deposited on the bioscaffold may be any suitable number. In some cases, the number of cells deposited on the polymeric scaffold is 10⁴ cells or more, e.g., 10⁵ cells or more, 10⁶ cells or more, including 10⁷ cells or more, and in some cases, may be 10¹² cells or less, e.g., 10¹¹ cells or less, 10¹⁰ cells or less, 10⁹ cells or less, 10⁸ cells or less, 10⁷ cells or less, including 10⁶ cells or less. In some embodiments, the number of cells deposited on the polymeric scaffold is in the range of 10⁴ cells to 10¹² cells, e.g., 10⁴ cells to 10¹⁰ cells, 10⁴ cells to 10⁸ cells, including 10⁴ cells to 10⁷ cells.

The cells deposited on the scaffold may be any suitable type of cells (e.g., therapeutic cells and/or precursors thereof, as described above). In some cases, the cells deposited on the scaffold are fully differentiated, therapeutic cells (e.g., primary cells, in vitro differentiated stem cells or precursor cells, etc.). In some cases, the cells deposited on the scaffold are undifferentiated stem cells, partially differentiated cells and/or precursor cells, and the cells may start or continue to differentiate while being cultured in the scaffold.

In some cases, the present method includes precoating the scaffold before depositing the cells. The scaffold may be precoated with a suitable coating material to facilitate attachment of the cells to the scaffold. The coating material may be any suitable biological of synthetic coating material, as described above. The scaffold may be coated with the coating material using any suitable method (e.g., by contacting the scaffold with a solution that contains an appropriate amount of the coating material, under suitable conditions). In some cases, the scaffold, with or without the coating material, is conditioned with the culture medium before depositing the cells (e.g., by contacting the scaffold, with or without the coating material, with the culture medium, under suitable conditions (such as at 37° C. for 6-18 hours).

### Utility

An intravascular catheter comprising a bioscaffold containing therapeutic cells will find many uses where it is desirable to transplant a population of therapeutic cells into an individual to treat a condition, e.g., a disease. As described herein, a variety of types of therapeutic cells (e.g., cells that secrete a hormone) can be loaded into the porous scaffold, which provides a microenvironment conducive for survival, growth and functional activity of the therapeutic cells in an in vivo environment of the transplant host. Upon placement of the catheter in a vein, the side holes in the catheter tube allow blood to flow though the catheter thereby supplying oxygen and nutrients to any loaded cellular cargo and also providing for the removal of waste products.

In some cases, where the therapeutic cells substantially remain in the bioscaffold within the intravascular catheter when implanted in a blood vessel, the therapeutic cells may be removed, if necessary, by removing the intravascular catheter from the subject. In such cases, the bioscaffold with the intravascular catheter may be designed (e.g., by providing an appropriate porosity) to retain the therapeutic cells in the bioscaffold over the desired duration of time. In some cases, the intravascular catheter is removed from the subject and replaced with another intravascular catheter to provide a bioscaffold with fresh therapeutic cells.

### Kits

Also provided herein is a kit comprising an intravascular catheter that can be used in performing methods of the present disclosure. In some embodiments, the assembled catheter comprising a bioscaffold with therapeutic cells is contained in a sterile package. Alternatively, the kit may contain the unassembled components of the intravascular catheter, including the catheter with or without the internal bioscaffold and the therapeutic cells contained in one or more sterile packages. If the kit provides the catheter without the bioscaffold within the catheter lumen, the kit may further comprise the reagents needed for preparing the bioscaffold (e.g., silicone elastomer, curing agent, porogen (e.g., NaCl), solvents, etc.). In some cases, the kit further comprises cells, e.g., therapeutic cells, or a precursor thereof, suitable for encapsulation within the bioscaffold of the intravascular catheter, as described herein. In some embodiments, the cells are encapsulated within the pores (e.g., macropores) of the bioscaffold inside the lumen of the intravascular catheter. In some cases, the kit further comprises an active agent, which may be included in the bioscaffold, therapeutic cells, or separately. The different components of the kit may be provided in separate containers, as appropriate.

The kit may also include a packaging that includes a compartment, e.g., a sterile compartment, for holding the intravascular catheter. The packaging may be any suitable packaging for holding the intravascular catheter. Examples of packaging and methods of packaging are described in, e.g., U.S. Pat. Nos. 3,755,042, 4,482,053, 4,750,619; U.S. App. Pub. Nos. 20050268573, 20100133133, each of which are incorporated herein by reference.

In addition to the above components, the subject kits may further include (in certain embodiments) instructions for practicing the subject methods. In some embodiments, instructions for using the intravascular catheter for implanting therapeutic cells in a subject are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-73 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. An intravascular catheter comprising:
a) a catheter tube comprising a lumen and a plurality of side holes, wherein the side holes are distributed along the length of the catheter tube in a spiraling corkscrew pattern; and
b) a biocompatible bioscaffold comprising a plurality of macropores and micropores, wherein the biocompatible bioscaffold is contained within the catheter lumen.

2. The intravascular catheter of aspect 1, wherein the catheter tube comprises polyurethane or silicone.

3. The intravascular catheter of aspect 1 or 2, wherein the bioscaffold comprises polydimethylsiloxane (PDMS), collagen, or graphene.

4. The intravascular catheter of any one of aspects 1 to 3, wherein the bioscaffold further comprises a coating comprising an anticoagulant.

5. The intravascular catheter of aspect 4, wherein the anticoagulant is heparin or warfarin.

6. The intravascular catheter of any one of aspects 1 to 5, wherein the catheter tube has a French size ranging from 3 french to 20 french.

7. The intravascular catheter of any one of aspects 1 to 6, wherein the side holes are about 0.2 to about 5.0 mm wide, about 3 mm to about 10 mm apart, and rotated about 30° to about 60° between each side hole.

8. The intravascular catheter of aspect 7, wherein the side holes are about 5.0 mm wide, about 10 mm apart, and rotated about 60° between each side hole.

9. The intravascular catheter of aspect 8, wherein the catheter tube has a French size of 14 french.

10. The intravascular catheter of aspect 7, wherein the side holes are about 0.2 mm wide, about 3.0 mm apart and rotated about 30° between each hole.

11. The intravascular catheter of aspect 10, wherein the catheter tube has a French size of 4 french.

12. The intravascular catheter of any one of aspects 1 to 11, wherein the bioscaffold has a porosity ranging from 30 percent to 95 percent.

13. The intravascular catheter of aspect 12, wherein the porosity is about 80 percent.

14. The intravascular catheter of any one of aspects 1 to 13, wherein the macropores have an average diameter ranging from about 150 μm to about 800 μm.

15. The intravascular catheter of aspect 14, wherein the macropores have an average diameter ranging from about 150 to about 250 μm

16. The intravascular catheter of any one of aspects 1 to 15, wherein the micropores have an average diameter of 30 μm or less.

17. The intravascular catheter of any one of aspects 1 to 16, wherein the bioscaffold further comprises one or more drugs, growth factors, angiogenic agents, cytokines, or extracellular matrix components, or a combination thereof.

18. The intravascular catheter of any one of aspects 1 to 17, wherein the bioscaffold further comprises therapeutic cells, wherein the therapeutic cells are contained in the macropores.

19. The intravascular catheter of aspect 18, wherein the therapeutic cells are stem cells, progenitor cells, or mature cells.

20. The intravascular catheter of aspect 19, wherein the stem cells are induced-pluripotent stem cells or adult stem cells.

21. The intravascular catheter of any one of aspects 18 to 20, wherein the therapeutic cells secrete a cytokine, a chemokine, a growth factor, or a hormone.

22. The intravascular catheter of aspect 21, wherein the therapeutic cells are endocrine cells.

23. The intravascular catheter of any one of aspects 18 to 22, wherein the therapeutic cells are genetically modified cells.

24. The intravascular catheter of any one of aspects 18 to 23, wherein the therapeutic cells are insulin-secreting cells.

25. The intravascular catheter of aspect 24, wherein the insulin-secreting cells are pancreatic beta cells or islets obtained from a donor.

26. The intravascular catheter of aspect 24, wherein the insulin-secreting cells are derived from stem cells or pancreatic progenitor cells.

27. A method of implanting therapeutic cells in a subject, the method comprising placing the intravascular catheter of any one of aspects 18 to 26 within a major vein of the subject.

28. The method of aspect 27, wherein the major vein is selected from the group consisting of an internal jugular vein, a subclavian vein, and a femoral vein.

29. The method of aspect 27 or 28, wherein the therapeutic cells are autologous, allogeneic, or xenogeneic.

30. The method of any one of aspects 27 to 29, further comprising retrieving the intravascular catheter from the subject and exchanging the intravascular catheter for another intravascular catheter comprising therapeutic cells.

31. A method of treating a subject for type 1 diabetes, the method comprising placing the intravascular catheter of any one of aspects 24 to 26 within a major vein of the subject.

32. The method of aspect 31, wherein the major vein is selected from the group consisting of an internal jugular vein, a subclavian vein, and a femoral vein.

33. The method of aspect 31 or 32, wherein the insulin-secreting cells are autologous, allogeneic, or xenogeneic pancreatic beta cells or islets.

34. The method of aspect 31 or 32, wherein the insulin-secreting cells are derived from stem cells or pancreatic progenitor cells.

35. The method of any one of aspects 31 to 34, further comprising retrieving the intravascular catheter from the subject and exchanging the intravascular catheter for another intravascular catheter comprising insulin-secreting cells.

36. A kit comprising:
a) the intravascular catheter of any one of aspects 1 to 17; and
b) a packaging comprising a compartment for holding the intravascular catheter.

37. The kit of aspect 36, further comprising therapeutic cells.

38. The kit of aspect 37, wherein the therapeutic cells are contained in the macropores of the biocompatible bioscaffold or in a container separate from the intravascular catheter.

39. The kit of aspect 37 or 38, wherein the therapeutic cells are stem cells, progenitor cells, or mature cells.

40. The kit of aspect 39, wherein the stem cells are induced-pluripotent stem cells or adult stem cells.

41. The kit of any one of aspects 37 to 40, wherein the therapeutic cells secrete a cytokine, a chemokine, a growth factor, or a hormone.

42. The kit of aspect 41, wherein the therapeutic cells are endocrine cells.

43. The kit of any one of aspects 37 to 42, wherein the therapeutic cells are genetically modified cells.

44. The kit of any one of aspects 37 to 43, wherein the therapeutic cells are insulin-secreting cells.

45. The kit of aspect 44, wherein the insulin-secreting cells are pancreatic beta cells or islets obtained from a donor.

46. The kit of aspect 44, wherein the insulin-secreting cells are derived from stem cells or pancreatic progenitor cells.

47. The kit of any one of aspects 37 to 46, further comprising instructions for using the intravascular catheter.

48. A method of producing the intravascular catheter of aspect 3, the method comprising:
a) combining polydimethylsiloxane (PDMS) with a porogen to produce a PDMS-porogen mixture;
b) placing the PDMS-porogen mixture inside the catheter lumen;
c) curing the PDMS-porogen mixture inside the catheter lumen;
d) adding a plurality of side holes to the catheter tube, wherein the side holes are distributed along the length of the catheter tube in a spiraling corkscrew pattern; and
e) removing the porogen from the PDMS-porogen mixture, wherein a PDMS bioscaffold comprising a plurality of macropores and micropores is produced inside the catheter lumen.

49. The method of aspect 48, wherein the porogen is water soluble.

50. The method of aspect 49, wherein the porogen is an alkali metal salt.

51. The method of aspect 50, wherein the porogen is sodium chloride or potassium chloride.

52. The method of any one of aspects 49 to 51, wherein said removing the porogen comprises submerging the intravascular catheter in water.

53. The method of any one of aspects 48 to 52, further comprising adding therapeutic cells to the bioscaffold, wherein the therapeutic cells are contained in the macropores.

54. The method of any one of aspects 48 to 53, wherein the catheter tube has a French size ranging from 3 french to 20 french.

55. The method of any one of aspects 48 to 54, wherein the side holes are about 0.2 to about 5.0 mm wide, about 3 mm to about 10 mm apart, and rotated about 30° to about 60° between each side hole.

56. The method of aspect 55, wherein the side holes are about 5.0 mm wide, about 10 mm apart, and rotated about 60° between each side hole.

57. The method of aspect 56, wherein the catheter tube has a French size of 14 french.

58. The method of aspect 55, wherein the side holes are about 0.2 mm wide, about 3.0 mm apart and rotated about 30° between each hole.

59. The method of aspect 58, wherein the catheter tube has a French size of 4 french.

60. The method of any one of aspects 48 to 59, wherein the bioscaffold has a porosity ranging from 30 percent to 95 percent.

61. The method of any one of aspects 48 to 60, wherein the catheter comprises polyurethane or silicone.

62. A method of producing an intravascular catheter, the method comprising:
forming a catheter tube comprising a plurality of side holes using three-dimensional (3D) printing, wherein the side holes are distributed along the length of the catheter tube in a spiraling corkscrew pattern; and
forming a biocompatible bioscaffold using the 3D printing, wherein the biocompatible bioscaffold is contained inside the catheter lumen.

63. The method of aspect 62, wherein the 3D-printing is used to generate the catheter tube and the biocompatible scaffold together or separately.

64. The method of aspect 62 or 63, wherein the bioscaffold comprises a geometric lattice inside the catheter tube.

65. The method of any one of aspects 62 to 64, wherein the bioscaffold comprises a plurality of macropores and micropores.

66. The method of aspect 65, wherein the macropores have an average diameter ranging from about 150 μm to about 800 μm.

67. The method of aspect 66, wherein the macropores have an average diameter ranging from about 150 to about 250 μm

68. The method of any one of aspects 65 to 67, wherein the micropores have an average diameter of 30 μm or less.

69. The method of any one of aspects 65 to 68, wherein the bioscaffold has a porosity ranging from 30 percent to 95 percent.

70. The method of any one of aspects 62 to 69, wherein the side holes are about 0.2 to about 5.0 mm wide, about 3 mm to about 10 mm apart, and rotated about 30° to about 60° between each side hole.

71. The method of any one of aspects 62 to 70, further comprising adding therapeutic cells to the bioscaffold.

72. The method of any one of aspects 62 to 71, wherein the catheter tube has a French size ranging from 3 french to 20 french.

73. The method of any one of aspects 62 to 72, further comprising designing a model of the intravascular catheter using computer aided designing (CAD) software to control production of the intravascular catheter by the 3D printing.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### A Hybrid PDMS Bioscaffold-Intravascular Catheter for Cellular Therapies

Tissue engineering approaches have been used in islet transplantation including using encapsulation strategies (micro- and microencapsulation systems) or three-dimensional (3D) porous bioscaffolds. While encapsulation of islets can prevent immune mediated attack, bioscaffolds can provide a 3D matrix to enable islet seeding without clumping while concurrently facilitating islet vascularization by the surrounding tissue.¹³ However, synthetic bioscaffolds can induce an intense inflammatory reaction which can have adverse effects on islet survival and function. In addition, there are only a few sites which can accommodate the volume of a bioscaffold.¹⁴

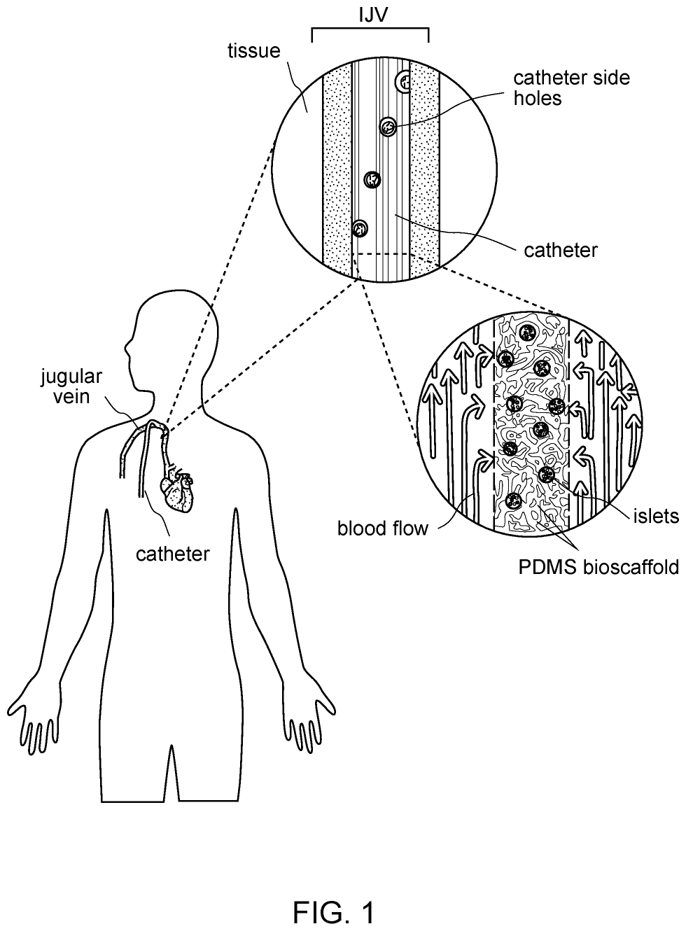

One solution to address the issues related to retrievability (which will become increasingly more important as stem cell derived beta cells gain traction as a source of transplantable insulin producing cells) and oxygen/nutrient availability is to create a system which can be placed directly into blood vessels. Hence, in the present study, we developed an intravascular multi-side hole catheter with an interior PDMS bioscaffold (FIG. 1). While the PDMS bioscaffold provides a structure to enable the even distribution of cells throughout the catheter, the catheter's placement in the blood vessels, and the ability of blood to flow though the catheter through side holes, will enable any loaded cellular cargo to be supplied with oxygen and nutrients while ensuring an efficient system for the removal of waste products. Hence, we used computational fluid dynamics (CFD) to develop a catheter design which we actualized by fabricating a PDMS bioscaffold within a multi-side hole silicone catheter.

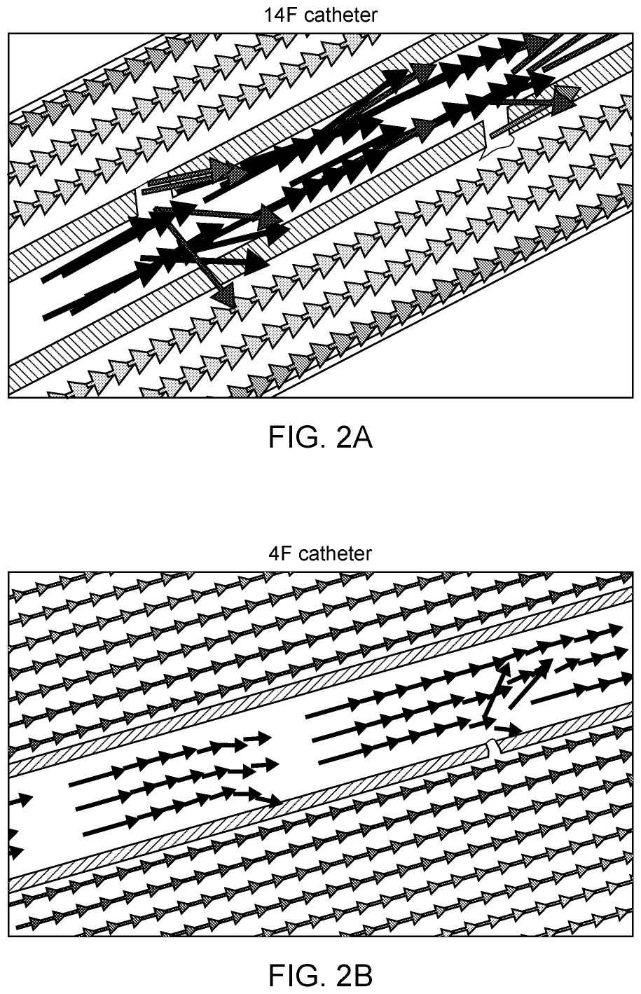

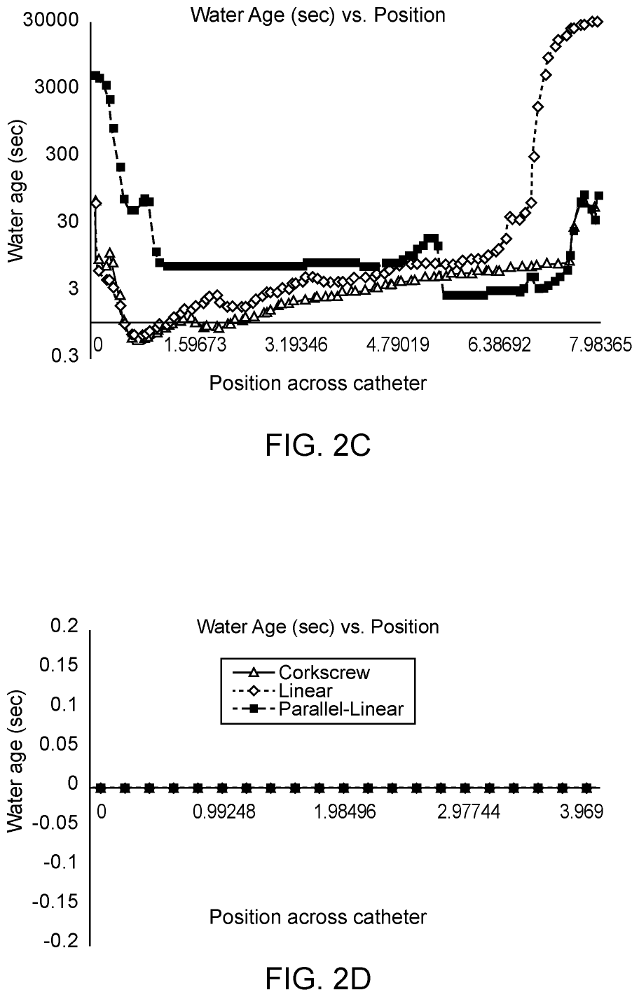

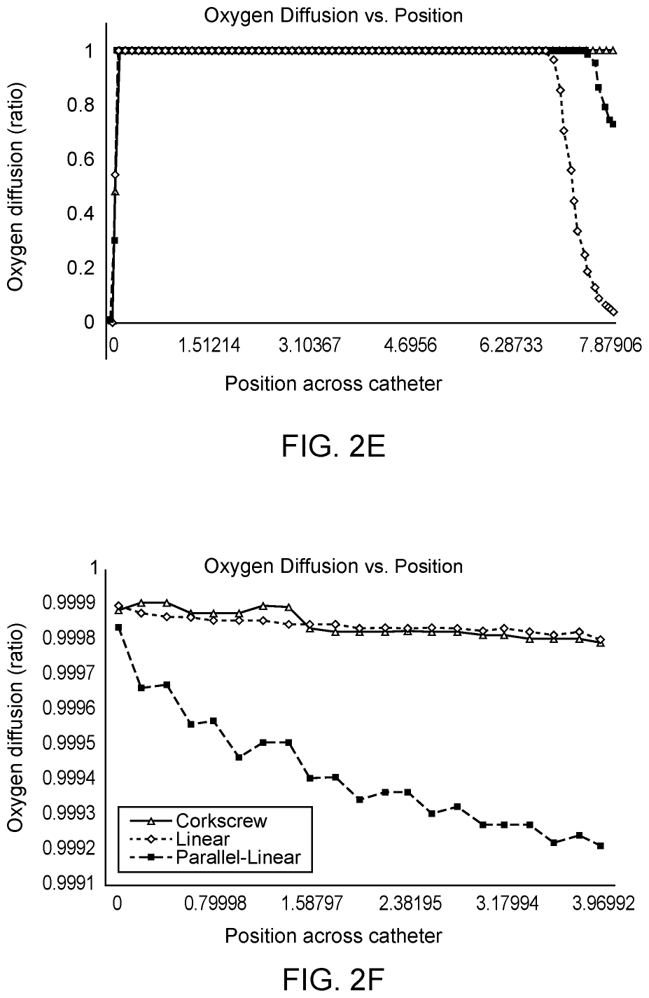

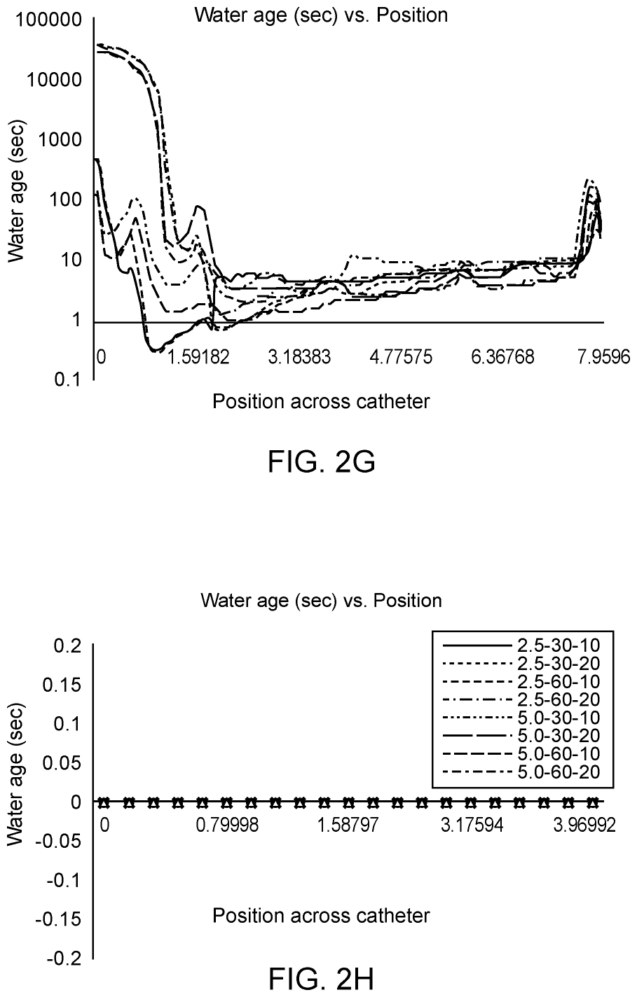

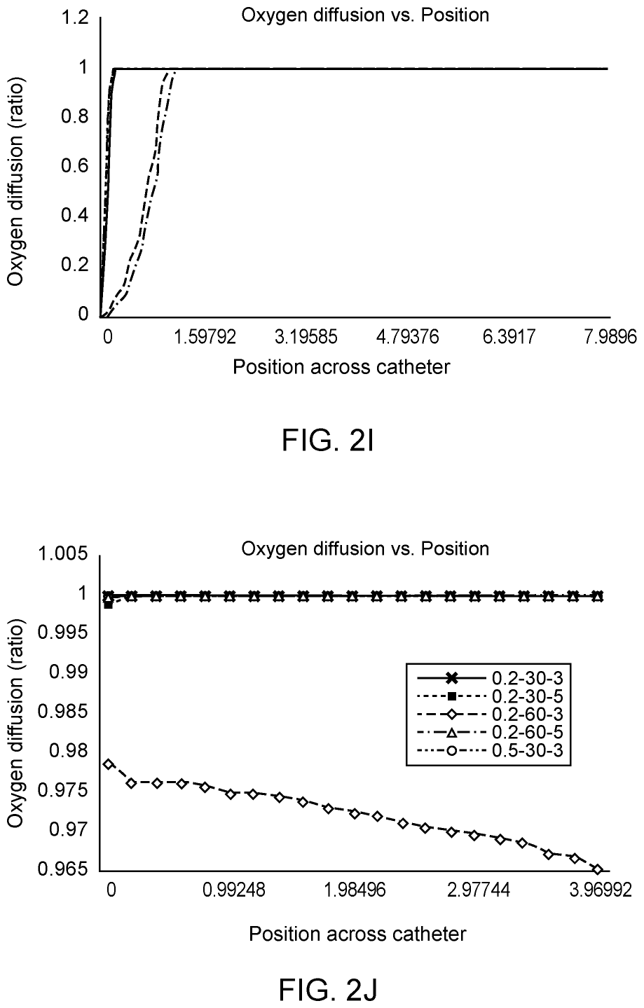

First, the optimal catheter design was determined by conducting CFD simulations on variations of 14F and 4F-sized catheters which would accommodate a range of differently sized patients.^{15,16} Both 14F and 4F size catheters were simulated for use in a blood vessel, such as the internal jugular vein. Simulations were evaluated for two parameters: i) water age within the model, indicating the degree to which blood might be expected to stay within the catheter and thereby increase chances of clotting, and ii) oxygen diffusion into the model, indicating the degree to which oxygen in blood might be expected to diffuse into the interior bioscaffold of the catheter. First, three different side hole orientations along the catheter shaft in both the 14F and 4F catheters were evaluated to determine the optimal side hole placement. The experimental orientations were a spiraling “corkscrew” distribution (corkscrew), two parallel linear lines of holes (parallel-linear), and a single linear line of holes (linear) serving as the control. The spiraling corkscrew distribution of side holes along the length of the catheter was found to confer the greatest flow into and out of both the 14F and 4F catheters by minimizing water age and maximizing oxygen diffusion in the catheter (FIG. 2, left). Using the corkscrew hole distribution pattern, the side hole diameter, distance between holes, and rotation angle of the holes along the length of the catheter (FIG. 2, right) were varied for both the 14F and 4F catheters.

The optimized 14F model that minimized water age and maximized oxygen diffusion in the catheter had side holes 5.0 mm wide, 10 mm apart and rotated 60° between each hole. Blood entered side holes at a rate of 12 cm/s and exited the side holes at a rate of 5.5-8 cm/s. On the other hand, the optimized 4F model that minimized water age and maximized oxygen diffusion had side holes 0.2 mm wide, 3.0 mm apart and rotated 30° between each hole. Blood entered the side holes at a rate of 4 cm/s and exited the side holes at a rate of 2-3.5 cm/s. Based on this data, the 4F catheter better minimized water age and maximized oxygen diffusion compared to the 14F catheter (FIG. 2). Furthermore, a 4F catheter is more clinically translatable as large catheters have been shown to cause excessive shear stress on blood vessel walls, which will be important for pediatric patients who have smaller caliber vessels.^{15,16} For the above reasons, the 4F catheter was selected for the following in vitro experiments. Our study demonstrates the ability of CFD simulations to develop catheter designs which included a resistance approximation to account for the complex bioscaffold matrix housed inside our catheters. This is in accordance with other work which have similarly used CFD to compare blood flow recirculation in hemodialysis catheters or evaluate obstructions in ventricular catheters used for the treatment of hydrocephalus.^{17,18} By experimenting with catheter sizes and designs, we were able to minimize the water age and maximize oxygen diffusion within the catheter. Ultimately, this should translate to shorter stagnation of blood flow which will prevent clotting within the catheter while maximizing the diffusion of oxygen to the cellular cargo housed within the bioscaffold matrix inside the catheter.

Next, we optimized the PDMS bioscaffold within a 4F catheter at 30%, 50%, 60%, 70%, and 80% porosities in order to provide a support matrix for islets to reside upon. SEM images revealed that optimal PDMS bioscaffold formation was at 80% porosity (FIG. 3A), where interconnected pores 150-250 μm in diameter were formed throughout the bioscaffold with minimal separation from the catheter wall (FIG. 3B). 80% porous PDMS bioscaffolds were then tested in vitro, using mouse islets as our chosen cellular therapy. In keeping with this, previous studies demonstrate that bioscaffolds with a higher porosity are ideal for islet growth given that they minimize cellular fibrosis and allow for greater cell-cell interactions within interconnected pores.^{19,20} Other studies typically fabricate polymeric bioscaffolds on the order of 5-30 mm in diameter in cylindrical molds. Contrary to this, our PDMS bioscaffolds were formed within the irregular confines of the catheter lumen and so our results demonstrate a flexible usage of solvent casting and particulate leaching techniques to create PDMS bioscaffolds within irregular and confined spaces.

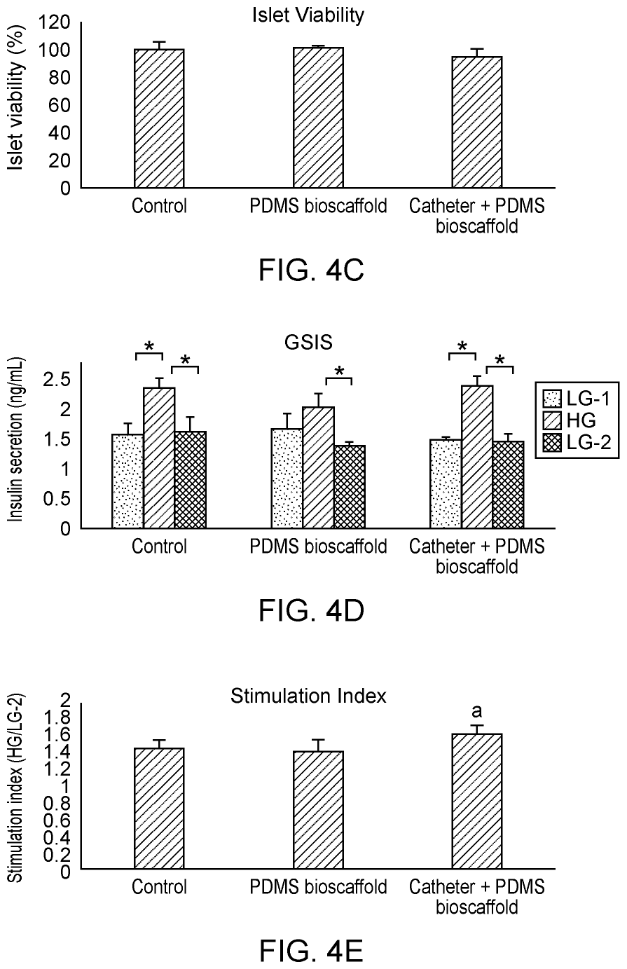

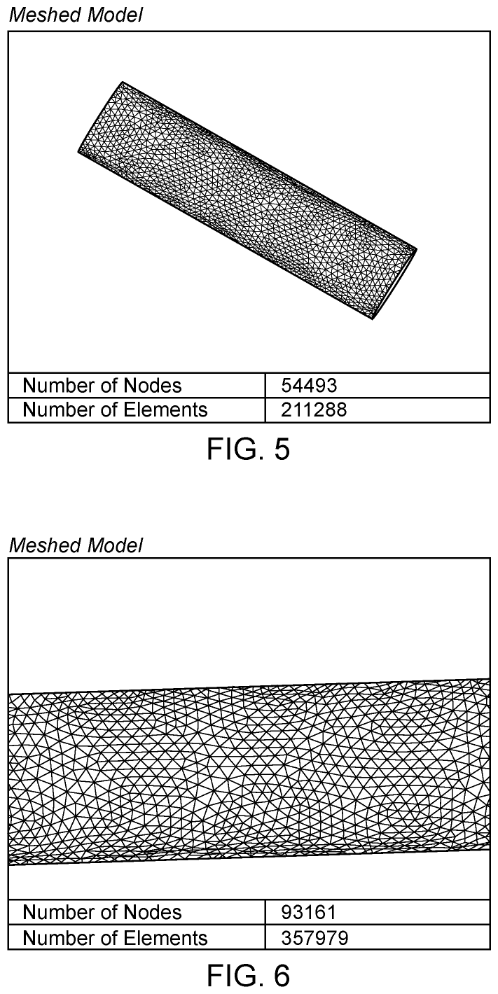

To evaluate the catheter's ability to maintain islet populations in vitro, we used a Live/Dead assay to assess islet viability and a glucose stimulated insulin secretion assay to assess islet function. For the Live/Dead assay, islet viability was measured with fluorescence microscopy. Islets alone served as the control group. Islets incubated within 1-cm sections of the catheter only (without PDMS), PDMS only (without the catheter), and catheter with PDMS served as the experimental groups. After 24 hours incubation, both control and experimental groups were stained with a solution of 84 of fluorescein diacetate (FDA, 5 mg/mL)+50 μL of propidium iodide (PI, 2 mg/mL) in a 96-well plate and incubated for 5 minutes. After standardizing the results to the control group, control group cell viability was 100.0±5.5%, PDMS bioscaffold only cell viability was 101.6±1.2%, and catheter containing the PDMS bioscaffold cell viability was 95.1±5.5%. One-way ANOVA demonstrated there was no significant difference in islet viability between the control and experimental groups (FIG. 4C).

Lastly, we used a GSIS assay to evaluate the ability of islets to respond to glucose changes in their different experimental conditions. Islets were incubated within 1-cm sections of the catheter only (without PDMS), PDMS only (without the catheter), and catheter with PDMS overnight. Then, each section was placed into a 12-well plate containing Transwell®. For the control group, islets were handpicked and placed directly into the top wells of the Transwell® insert. Both the control and the experimental groups were incubated with low glucose (2.3 mM glucose in Krebs Ringer Bicarbonate Buffer (13.7 mM NaCl, 4.7 mM KCl, 1.2 mM KH₂PO₄, 1.2 mM MgSO₄-7H₂O, 2.5 mM CaCl₂)-2H₂O, 25 mM NaHCO₃), LG-1) in the bottom well of the Transwell® system.

After 1 hour, the supernatant in the bottom compartment was collected and immediately stored in −80° C. This process was repeated with high glucose (20 mM glucose in Krebs Ringer Bicarbonate Buffer, HG) and low glucose (2.3 mM glucose in Krebs Ringer Bicarbonate Buffer, LG-2). Islets seeded in PDMS bioscaffold with or without a catheter showed well-preserved glucose responsiveness, demonstrated as increased insulin release upon glucose stimulation at high concentration (HG, 20.0 mM of glucose) and a subsequent decrease when incubated in low glucose buffer (LG2, 2.3 mM of glucose) (FIGS. 4D, 4E), when compared to control islets (free-floating islets). This behavior was highlighted in FIG. 4E, where GSIS is expressed as insulin stimulation index (SI: insulin secreted during high glucose divided by release during low glucose). The PDMS bioscaffold did not affect the glucose responsiveness of the islets, and furthermore insulin secretion increased for islets within the catheter containing the PDMS bioscaffold (SI=1.63±0.10) compared to islets within the PDMS bioscaffold (SI=1.40±0.14, p<0.05) and free-floating islets (SI=1.46±0.10). In vitro analysis demonstrated the ability of our hybrid PDMS bioscaffold-intravascular catheter to facilitate normal islet survival and function. Our Live/Dead assay demonstrated that the hybrid PDMS bioscaffold-intravascular catheter maintained pancreatic islet survival, in keeping with previous studies that found PDMS as a highly biocompatible and frequently used biomaterial in various devices.

Advantages of PDMS bioscaffolds include that they are inert to most mild thermal and oxidative conditions, transparent, permeable to small vapor and gas molecules (i.e. water and oxygen), and have good dielectric properties.²¹⁻²³ They have also been shown to have good long-term durability (up to 32 years after implantation). Furthermore, the porous structure of the PDMS bioscaffold not only allows cells like islets to be housed and evenly distributed throughout the macropores, but it also facilitates the formation of a vascular network through the bioscaffold via its micropores. Indeed, islets seeded into a catheter without the bioscaffold led to cellular clumping and reduced viability due to the cells/islets in the center of the clump dying from hypoxia and nutrient deprivation, hence an internal bioscaffold structure which can support islets and facilitate their distribution through the catheter is needed.

The GSIS data also demonstrates that the hybrid PDMS bioscaffold-intravascular catheter can maintain glucose responsiveness compared to islets alone. This was an improvement compared to islets cultured on PDMS bioscaffolds alone and is possibly due to the additional support that the catheter provides by containing all the islets within the hybrid PDMS bioscaffold-catheter construct. Currently, islets are transplanted by injecting them directly into the portal vein where they then engraft in the liver—here, they are subjected to a relatively hostile microenvironment with localized inflammation and low oxygen tensions resulting in their loss of function and survial.^{24,25} Using our hybrid catheter, islets can now be placed directly in the bloodstream so they have a continuous and readily available supply of nutrients and oxygen, while also being able to effectively sample circulating glucose levels, without being subjected to tissue mediated inflammatory reactions.

Current technologies have sought to promote more hyperoxic sites of transplantation instead; for example, Beta O2 Technologies created a small, subcutaneously implanted cell encapsulation disk that houses 2,000 islet equivalents and produces insulin that diffuses across an alginate membrane. However, this disk must receive a daily resupply of oxygen (40% oxygen and 5% CO₂) via a tube that extends outside of the body, requiring the user to regularly supplement themselves with oxygen.²⁶ Our hybrid catheters can be simply implanted using conventional techniques and also retrieved or exchanged easily. This is an advantage over other devices' given their issues with retrievability.²⁷ For example, Cerco Medical developed an alginate sheet containing islets but encountered issues finding an optimal insertion site that could accommodate all the sheets while providing enough nutrients and oxygen to islets. The alginate sheets also became highly vascularized by the surrounding tissue which would again prove problematic if the sheet needed to be retrieved.²⁸ Viacyte created a retrievable subcutaneous disk containing islet progenitor cells that mature within the body, but there are questions as to the extent to which these cells can mature and produce an adequate amount of insulin. Future studies of this catheter will therefore start to examine the feasibility of translating this catheter into diabetic large animal models where it can be tested with either islets or stem cell-derived beta cells.

In summary, this work is the proof-of-concept for a novel hybrid PDMS bioscaffold intravascular catheter designed to be placed directly into the bloodstream, bypassing issues related to hypoxia at traditional sites of transplantation. This catheter was developed with CFD and bioscaffold engineering and assessed in vitro for its ability to maintain the survival and function of pancreatic islets. This catheter offers a clinically translatable platform for islet transplantation for T1 D treatment as well as for other cellular therapies following validation in large animal models.

### REFERENCES

WHO| Diabetes. WHO. 2017. Center for Disease Control. National Diabetes Statistic Report, 2017. CDC.gov.cdc.gov/features/diabetes-statistic-report/index.html. Published 2017. Accessed Mar. 1, 2018.
Primavera R, Kevadiya B D, Swaminathan G, et al. Emerging Nano- and Micro-Technologies Used in the Treatment of Type-1 Diabetes. Nanomaterials. 2020; 10(4):789. doi:10.3390/nano10040789
Veiseh O, Tang B C, Whitehead K A, Anderson D G, Langer R. Managing diabetes with nanomedicine: challenges and opportunities. Nat Rev Drug Discov. 2015; 14(1):45-57. doi:10.1038/nrd4477
Chu L Y. Controlled release systems for insulin delivery. Expert Opin Ther Pat. 2005; 15(9):1147-1155. doi:10.1517/13543776.15.9.1147
Di J, Price J, Gu X, Jiang X, Jing Y, Gu Z. Ultrasound-Triggered Regulation of Blood Glucose Levels Using Injectable Nano-Network. Adv Healthc Mater. 2014; 3(6):811816. doi:10.1002/adhm.201300490
Fiorina P, Shapiro A M J, Ricordi C, Secchi A. The Clinical Impact of Islet Transplantation. Am J Transplant. 2008; 8(10):1990-1997. doi:10.1111/j.1600-6143.2008.02353.x
Liew A, O'Brien T. The Potential of Cell-based Therapy for Diabetes and Diabetes-related Vascular Complications. Curr Diab Rep. 2014; 14(3):469. doi:10.1007/s11892-013-0469-6
Foster E D, Bridges N D, Feurer I D, et al. Improved Health-Related Quality of Life in a Phase 3 Islet Transplantation Trial in Type 1 Diabetes Complicated by Severe Hypoglycemia. Diabetes Care. 2018; 41(5):1001-1008. doi:10.2337/dc17-1779
Muthyala S, Safley S, Gordan K, Barber G, Weber C, Sambanis A. The effect of hypoxia on free and encapsulated adult porcine islets—an in vitro study. Xenotransplantation 2017; 24(1):e12275. doi:10.1111/xen.12275
Geleto G, Getnet W, Tewelde T. Mean Normal Portal Vein Diameter Using Sonography among Clients Coming to Radiology Department of Jimma University Hospital, Southwest Ethiopia. Ethiop J Health Sci. 2016; 26(3):237-242. doi:10.4314/ejhs.v26i3.6
Goldstick T K, Ciuryla V T, Zuckerman L. Diffusion of oxygen in plasma and blood. Adv Exp Med Biol. 1976; 75:183-190.
Gibly R F, Zhang X, Lowe W L, Shea L D, Shea L D. Porous scaffolds support extrahepatic human islet transplantation, engraftment, and function in mice. Cell Transplant. 2013; 22(5):811-819. doi:10.3727/096368912X636966
Saleh L S, Bryant S J. The host response in tissue engineering: Crosstalk between immune cells and cell-laden scaffolds. Curr Opin Biomed Eng. 2018; 6:58-65. doi:10.1016/j.cobme.2018.03.006
Kinney T B, Fan M, Chin A K, Finn J C, Hayden W G, Fogarty T J. Shear Force in Angioplasty: Its Relation to Catheter Design and Function. Am J Roentgenol. 1985; 144:115-122.
Piper R, Carr P J, Kelsey L J, Bulmer A C, Keogh S, Doyle B J. The mechanistic causes of peripheral intravenous catheter failure based on a parametric computational study. Sci Rep. 2018; 8(1). doi:10.1038/s41598-018-21617-1
Clark T W I, Isu G, Gallo D, Verdonck P, Morbiducci U. Comparison of Symmetric Hemodialysis Catheters Using Computational Fluid Dynamics. J Vasc Interv Radiol. 2015; 26(2):252-259.e2. doi:10.1016/J.JVIR.2014.11.004
Galarza M, Giménez Á, Valero J, Pellicer O P, Amigó J M. Computational fluid dynamics of ventricular catheters used for the treatment of hydrocephalus: A 3D analysis. Child's Nerv Syst. 2014; 30(1):105-116. doi:10.1007/500381-013-2226-1
Pedraza E, Brady A C, Fraker C A, et al. Macroporous three-dimensional PDMS scaffolds for extrahepatic islet transplantation. Cell Transplant. 2013; 22(7):1123-1135. doi:10.3727/096368912X657440
Jiang K, Weaver J D, Li Y, Chen X, Liang J, Stabler C L. Local release of dexamethasone from macroporous scaffolds accelerates islet transplant engraftment by promotion of anti-inflammatory M2 macrophages. Biomaterials. 2017; 114:7181. doi:10.1016/j.biomaterials.2016.11.004
Hacker M C, Krieghoff J, Mikos A G. Synthetic Polymers. In: Principles of Regenerative Medicine. Elsevier; 2019:559-590. doi:10.1016/13978-0-12-809880-6.00033-3
Brandon H J, Jerina K L, Wolf C J, Young V L. Biodurability of retrieved silicone gel breast implants. Plast Reconstr Surg. 2003; 111(7):2295-2306. doi:10.1097/01.PRS.0000060795.16982.1C
Merkel T C, Bondar V I, Nagai K, Freeman B D, Pinnau I. Gas sorption, diffusion, and permeation in poly(dimethylsiloxane). J Polym Sci Part B Polym Phys. 2000; 38(3):415-434. doi:10.1002/(SICI)1099-0488(20000201)38:3<415::AID-POLB8>3.0.CO; 2-Z
Merani S, Toso C, Emamaullee J, Shapiro A M J. Optimal implantation site for pancreatic islet transplantation. Br J Surg. 2008; 95(12):1449-1461. doi:10.1002/bjs.6391
Carlsson P O, Palm F, Andersson A, Liss P. Markedly decreased oxygen tension in transplanted rat pancreatic islets irrespective of the implantation site. Diabetes. 2001; 50(3):489-495. doi:10.2337/diabetes.50.3.489
Barkai U, Weir G C, Colton C K, et al. Enhanced Oxygen Supply Improves Islet Viability in a New Bioartificial Pancreas. Cell Transplant. 2013; 22(8):1463-1476. doi:10.3727/096368912X657341
O'Sullivan E S, Vegas A, Anderson D G, Weir G C. Islets Transplanted in Immunoisolation Devices: A Review of the Progress and the Challenges that Remain. Endocr Rev. 2011; 32(6):827-844. doi:10.1210/er.2010-0026
Cerco Medical: R&D: Development of ISM's Thin Sheet. Accessed Mar. 1, 2018. hanumanmedical.com/pages/rnd_development.htm

### Example 2

### Materials and Methods

### Computational Fluid Simulation Studies

3D models of the hybrid PDMS bioscaffold-intravascular catheter were designed using Solidworks (2017) and Autodesk Inventor Professional (2018). Fluid dynamics were evaluated using Autodesk CFD (2017). We simulated both 14F and 4F size catheters for use in the internal jugular vein [23, 24]. The 14F catheter model was constructed with a 4.0 mm inner lumen diameter and 4.6 mm outer lumen diameter and the blood vessel was represented as an extruded cylinder of 20 mm surrounding the catheter. The 4F model was constructed with a 1.2 mm inner lumen diameter and 1.5 mm outer lumen diameter and the blood vessel was simulated as an extruded cylinder of 20 mm surrounding the catheter. The catheter and blood vessel were assigned the materials of silicon rubber and blood, respectively, in Autodesk CFD. The bioscaffold was simulated as an internal cylinder tangent to the lumen face of the catheter; the porous nature of the bioscaffold was modeled as a resistance to fluid flow with a flow coefficient=0.8. Boundary conditions included: 20 cm/s flow velocity, oxygen diffusion coefficient=1.6 e−5 (using a general scalar assigning the blood and bioscaffold of relative values 1 and 0, respectively), and exit pressure=0 mmHg (simulating low venous pressure) 17. CFD parameters can be found in Tables 2-5. We solved the system under non-Newtonian blood flow conditions for water age and oxygen diffusion across the length of the catheter to determine the best model. Simulations were run for 100 iterations with approximately 120,000 mesh elements per model.

### Catheter Materials

Medical-grade polyurethane catheters (Nordson MEDICAL, New Hampshire, USA) were used for all experiments. To provide a support matrix for islets, PDMS bioscaffolds were synthesized within the catheter via solvent casting and particulate leaching technique. First, a silicone elastomer base (RTV 615A) was mixed with the curing agent (RTV615B) in a 4:1 ratio (Momentive Performance Materials). This was then mixed with sodium chloride (150 μm-250 μm. particles, Fisher Chemical) to form 30, 50, 60, 70, and 80% bioscaffold porosities. The PDMS-salt mixture was pushed into the catheter and placed into an oven (Thermo Scientific, Heratherm Oven) at 90° C. for 7 days to cure the PDMS-salt mixture. After curing, side holes were punctured into the catheter using a soldering tip, and then the catheter was placed into a water shaker (Thermo Scientific, MaxQ 5000) at 200 rpm for 7 days at 37° C. to dissolve the salt particles and form a porous bioscaffold. Water was exchanged every day to ensure maximum salt dissolution. Bioscaffold porosity and homogeneity were evaluated through scanning electron microscopy (SEM) images (Stanford Nano Shared Facilities).

### Islet Isolation and Culture

Pancreatic islets were isolated from C57BL/6 mice using collagenase digestion and Histopaque® gradients solutions (1.119 g/mL and 1.077 g/mL for Histopaque®-1119 and Histopaque®-1077, respectively), as previously described¹⁸. In brief, the mouse pancreas was surgically exposed and the pancreatic duct isolated and cannulated. The pancreas was distended using an infusion of 2-3 mL of Hank's balanced salt solution (HBSS, Sigma-Aldrich) supplemented with 0.1% bovine serum albumin (BSA; Sigma-Aldrich) containing 1 mg/mL of collagenase VI (Sigma-Aldrich). The pancreas was then dissected and incubated for 10 min in a water bath at 37° C. Islets were purified by gradient centrifugation on Histopaque®-1119 and Histopaque®-1077 (Sigma-Aldrich), and then individually handpicked and cultured in culture petri dishes containing RPMI 1640 medium without Phenol Red supplemented with 10% fetal bovine serum (FBS).

### Cell Seeding

Mouse islets were seeded into the hybrid PDMS bioscaffold-intravascular catheters via pipetting. Specifically, islets were viewed under a stereomicroscope and handpicked with a 504 pipette to ensure an even distribution of sizes, and then housed into the catheter only (without PDMS), PDMS bioscaffold only (without the catheter), or the catheter containing the PDMS bioscaffold.

### Live/Dead Assay

Islets in all conditions were incubated for 24 hours in a 96-well plate (20 islets/well). Islet viability was measured with fluorescence microscopy using a live/dead assay. Islets alone served as the control group. After 24 hours incubation, both control and experimental groups were stained with a solution of 8 μL of fluorescein diacetate (FDA, 5 mg/mL)+50 μL of propidium iodide (PI, 2 mg/mL) in a 96-well plate and incubated for 5 minutes. They were then imaged using fluorescence microscopy (Leica Microscopy, California, USA) to assess islet viability. Live/dead analysis was conducted using Image J colorimetric quantification.

### Glucose Stimulated Insulin Sensitivity Assay

The functionality of islets seeded within hybrid PDMS bioscaffold-intravascular catheters were tested by examining their insulin secretion profiles using a glucose stimulated insulin secretion (GSIS) assay. A 12-well plate containing Transwell® with 0.4 μm pore polyester membrane inserts (Corning®, USA) was used to prevent islets from being inadvertently aspirated when changing glucose solutions (n=5). The Transwell® inserts were primed with 100 μL cell culture medium (RPMI without Phenol Red, 10% FBS) 30 minutes prior to beginning the GSIS assay. After isolation, islets were incubated overnight and then the specified number of islets (20 islets) were hand-picked up under stereomicroscopy using a 50 μL pipettor. Islets were incubated in 1 cm sections of the catheter only (without PDMS), PDMS bioscaffold only (without the catheter), and the catheter containing the PDMS bioscaffold. Each section was placed into the top well of the Transwell® insert. For the control group, islets were handpicked and placed directly into the top wells of the Transwell® insert achieving 20 islets in 200 μL of complete medium (RPMI without Phenol Red, 10% fetal bovine serum (FBS; Invitrogen, USA)). Both the control and the experimental groups were incubated with 200 μL of low glucose (2.3 mM glucose in Krebs Ringer Bicarbonate Buffer (137 mM NaCl, 4.7 mM KCl, 1.2 mM KH₂PO₄, 1.2 mM MgSO₄-7H₂O, 2.5 mM CaCl₂)-2H₂O, 25 mM NaHCO₃), LG-1) in the bottom well of the Transwell® system at 37° C. and 5% CO₂. After 1 hour, the supernatant in the bottom compartment was collected and immediately stored in −80° C. This process was repeated with high glucose (20 mM glucose in Krebs Ringer Bicarbonate Buffer, HG) and low glucose (2.3 mM glucose in Krebs Ringer Bicarbonate Buffer, LG-2). The supernatant was analyzed using the Mercodia Mouse Insulin ELISA kit.

### Statistical Analysis

All experiments were performed in n=5, and the results expressed as the mean±standard error of the mean. The statistical analysis was performed using a Student's t-test or one-way ANOVA with post-hoc Tuckey test. Differences were considered statistically significant with p<0.05.

TABLE 1
Materials

| Material | Properties | |
|---|---|---|
| Blood | Density | 1003.0 kg/m³ |
| | Viscosity | NonNewt Pwr Law |
| | Conductivity | 0.6 W/m-K |
| | Specific heat | 4182.0 J/kg-K |
| | Compressibility | 2185650000.0 Pa |
| | Emissivity | 0.7 |
| | Wall roughness | 0.0 meter |
| | Phase | Vapor Pressure |
| Silicon Rubber | X-Direction | 0.7 W/m-K |
| | Y-Direction | Same as X-dir. |
| | Z-Direction | Same as X-dir. |
| | Density | 1.7 g/cm³ |
| | Specific heat | 0.7 J/g-K |
| | Emissivity | 0.9 |
| | Transmissivity | 0.0 |
| | Electrical resistivity | 0.0 ohm-cm |
| | Wall roughness | 0.0 meter |
| Default Resistance | Through-Flow K | Free Area Ratio |
| | Normal direction 1K | 0.0 |
| | Normal direction 2K | 0.0 |
| | Conductivity | 1.0 W/m-K |

TABLE 2
Boundary conditions

| Type | Assigned to |
|---|---|
| Velocity | Blood vessel inlet. Models the velocity |
| Normal (20 cm/s) | of blood in the internal jugular vein. |
| Scalar (1) | Blood outside the catheter. Models the |
| | initial oxygenation of blood outside the catheter. |
| Pressure | Blood vessel inlet. Models the exit pressure |
| (0 dyne/cm2 Gage) | of the blood vessel, letting blood exit the vessel. |
| Velocity | Blood vessel outlet. Allows blood to exit the vessel. |
| Normal (0 cm/s) | |
| Scalar (0) | Blood inside the catheter. Models the initial |
| | unoxygenated state of blood inside the catheter. |
| Free Area | Resistance inside the catheter. Models the porosity |
| Ratio (.8) | of the bioscaffold inside the catheter. |

TABLE 3
Physics and solver settings

| Compressibility | Incompressible |
|---|---|
| Heat Transfer | Off |
| Radiation | Off |
| Scalar | General scalar |
| Turbulence | On |
| Solution mode | Steady State |
| Intelligent solution control | On |
| Advection scheme | ADV 1 |
| Turbulence model | k-epsilon |
| Iterations run | 100 |

TABLE 4
Mesh settings (14F)
Automatic Meshing Settings

| | |
|---|---|
| Surface refinement | 0 |
| Gap refinement | 0 |
| Resolution factor | 0.8 |
| Edge growth rate | 1.1 |
| Minimum points on edge | 2 |
| Points on longest edge | 10 |
| Surface limiting aspect ratio | 20 |

Mesh Enhancement Settings

| | |
|---|---|
| Mesh enhancement | 1 |
| Enhancement blending | 0 |
| Number of layers | 3 |
| Layer factor | 0.45 |
| Layer gradation | 1.05 |

TABLE 5
Mesh settings (4F)
Automatic Meshing Settings

| | |
|---|---|
| Surface refinement | 0 |
| Gap refinement | 0 |
| Resolution factor | 0.7 |
| Edge growth rate | 1.1 |
| Minimum points on edge | 2 |
| Points on longest edge | 10 |
| Surface limiting aspect ratio | 20 |

Mesh Enhancement Settings

| | |
|---|---|
| Mesh enhancement | 1 |
| Enhancement blending | 0 |
| Number of layers | 3 |
| Layer factor | 0.45 |
| Layer gradation | 1.05 |

1. An intravascular catheter comprising:
a) a catheter tube comprising a lumen and a plurality of side holes, wherein the side holes are distributed along the length of the catheter tube in a spiraling corkscrew pattern; and
b) a biocompatible bioscaffold comprising a plurality of macropores and micropores, wherein the biocompatible bioscaffold is contained within the catheter lumen.

2. The intravascular catheter of claim 1, wherein the catheter tube comprises polyurethane or silicone.

3. The intravascular catheter of claim 1, wherein the bioscaffold comprises polydimethylsiloxane (PDMS), collagen, or graphene.

4. The intravascular catheter of claim 1, wherein the bioscaffold further comprises a coating comprising an anticoagulant.

5-6. (canceled)

7. The intravascular catheter of claim 1, wherein the side holes are about 0.2 to about 5.0 mm wide, about 3 mm to about 10 mm apart, and rotated about 30° to about 60° between each side hole.

8-13. (canceled)

14. The intravascular catheter of claim 1, wherein the macropores have an average diameter ranging from about 150 μm to about 800 μm, the micropores have an average diameter of 30 μm or less, and the bioscaffold has a porosity ranging from 30 percent to 95 percent.

15-16. (canceled)

17. The intravascular catheter of claim 1, wherein the bioscaffold further comprises one or more drugs, growth factors, angiogenic agents, cytokines, therapeutic cells, or extracellular matrix components, or a combination thereof.

18. (canceled)

19. The intravascular catheter of claim 17, wherein the therapeutic cells are stem cells, progenitor cells, mature cells, or genetically modified cells.

20. (canceled)

21. The intravascular catheter of claim 17, wherein the therapeutic cells secrete a cytokine, a chemokine, a growth factor, or a hormone.

22-23. (canceled)

24. The intravascular catheter of claim 17, wherein the therapeutic cells are insulin-secreting cells.

25-26. (canceled)

27. A method of implanting therapeutic cells in a subject, the method comprising placing the intravascular catheter of claim 17 within a major vein of the subject.

28. The method of claim 27, wherein the major vein is selected from the group consisting of an internal jugular vein, a subclavian vein, and a femoral vein.

29. (canceled)

30. The method of claim 27, further comprising retrieving the intravascular catheter from the subject and exchanging the intravascular catheter for another intravascular catheter comprising therapeutic cells.

31. A method of treating a subject for type 1 diabetes, the method comprising placing the intravascular catheter of claim 24 within a major vein of the subject.

32. (canceled)

33. The method of claim 31, wherein the insulin-secreting cells are autologous, allogeneic, or xenogeneic pancreatic beta cells or islets, or insulin-secreting cells derived from stem cells, or pancreatic progenitor cells.

34. (canceled)

35. The method of claim 31, further comprising retrieving the intravascular catheter from the subject and exchanging the intravascular catheter for another intravascular catheter comprising insulin-secreting cells.

36-47. (canceled)

48. A method of producing the intravascular catheter of claim 3, the method comprising:
a) combining polydimethylsiloxane (PDMS) with a porogen to produce a PDMS-porogen mixture;
b) placing the PDMS-porogen mixture inside the catheter lumen;
c) curing the PDMS-porogen mixture inside the catheter lumen;
d) adding a plurality of side holes to the catheter tube, wherein the side holes are distributed along the length of the catheter tube in a spiraling corkscrew pattern; and
e) removing the porogen from the PDMS-porogen mixture, wherein a PDMS bioscaffold comprising a plurality of macropores and micropores is produced inside the catheter lumen.

49-54. (canceled)

55. The method of claim 48, wherein the side holes are about 0.2 to about 5.0 mm wide, about 3 mm to about 10 mm apart, and rotated about 30° to about 60° between each side hole.

56-61. (canceled)

62. A method of producing an intravascular catheter, the method comprising:
forming a catheter tube comprising a plurality of side holes using three-dimensional (3D) printing, wherein the side holes are distributed along the length of the catheter tube in a spiraling corkscrew pattern; and
forming a biocompatible bioscaffold using the 3D printing, wherein the biocompatible bioscaffold is contained inside the catheter lumen.

63. (canceled)

64. The method of claim 62, wherein the bioscaffold comprises a geometric lattice inside the catheter tube.

65-69. (canceled)

70. The method of claim 62, wherein the side holes are about 0.2 to about 5.0 mm wide, about 3 mm to about 10 mm apart, and rotated about 30° to about 60° between each side hole.

71-72. (canceled)

73. The method of claim 62, further comprising designing a model of the intravascular catheter using computer aided designing (CAD) software to control production of the intravascular catheter by the 3D printing.
